(19) United States
(12) Patent Application Publication
(10) Pub. No.: US 2003/0022292 A1
(43) Pub. Date: Jan. 30, 2003

(54) Ligation of CEACAM1

(76) Inventors: Scott D. Gray-Owen, Oakville (CA); Ian C. Boulton, Toronto (CA)

Correspondence Address: BERESKIN AND PARR, SCOTIA PLAZA, 40 KING STREET WEST-SUITE 4000 BOX 401, TORONTO, ON, M5H 3Y2, CA

(21) Appl. No.: 10/163,638

(22) Filed: Jun. 7, 2002

Related U.S. Application Data
(60) Provisional application No. 60/296,152, filed on Jun. 7, 2001.

(51) Int. Cl. C12P 21/06; A61K 39/00; A61K 39/38

(52) U.S. Cl. 435/69.1; 424/184.1

(57) ABSTRACT

Methods and compositions for suppressing an immune response and for inhibiting tumor cell growth are described. The methods involve ligating CEACAM1 using a neisserial Opa protein.

This application claims the benefit under 35 USC §119(e) from U.S. Provisional patent application serial No. 60/296,152, filed Jun. 7, 2001.

### FIELD OF THE INVENTION

The present invention relates to novel methods and compositions for modulating an immune response and for suppressing tumor cell growth.

### BACKGROUND OF THE INVENTION

Despite the availability of effective antibiotic therapies to combat infection, Neisseria gonorrhoeae causes ˜78 million infections globally per annum (World Health Organisation. http://www/who/int/whr/1995/state.html). Gonorrhea is characterized by an intense inflammatory response that leads to the liberation of large amounts of urethral or cervical pus, consisting primarily of neutrophils with extracellular and intracellular-associated N. gonorrhoeae. Despite this fact, up to 15% of infected men and 80% of infected women remain asymptomatic (1). In such situations, infection tends to be prolonged and is consistently transmissible, both vertically (to neonates of infected mothers) and horizontally (to sexual partners). If undetected, such infections are a source of significant morbidity, including conjunctivitis in neonates, disseminated gonococcal infection, pelvic inflammatory disease and sterility through fallopian tube scarring (2).

The persistence of N. gonorrhoeae within the population relies on the fact that gonorrhea can be contracted repeatedly, and there is little evidence that the infection reduces an individual's susceptibility to subsequent infection (1). This is at least partially attributable to the antigenic variation of gonococcal surface epitopes (3), however, individuals can be reinfected by the same serotype of N. gonorrhoeae (4-6) indicating that immunoevasion is not the only survival strategy used by this pathogen. While gonococci-specific immunoglobulins can be detected in serum and mucosal secretions, their concentration is typically low and short-lived (7,8). Furthermore, the antibody response that does occur is not protective, and is not higher during subsequent gonococcal infections, suggesting that immunological memory is not induced (8). These phenomenon are unlikely to result from a general inability to develop an immune response within the urogenital tract, since significant vaginal and cervical antibody responses can be generated by intravaginal immunization with appropriate epitopes (9). Moreover, the antibody response to gonococcal infection of the rectum, which contains lymphoid follicles that resemble Peyer's patches, is also weak (8). It thus appears likely that N. gonorrhoeae possess some mechanism by which to subvert the natural immune response. Such an immunosuppressive effect would also help to explain other clinical observations. For example, there is a transient decline in CD4⁺ T cell counts (10) and CD8⁺ T cell responses (R. Kaul et al., unpublished data) in blood during gonococcal infection, which resolves following clearance of the bacterial infection. Whether these effects help to explain why gonococcal infection also increases an individual's susceptibility to subsequent infection by both Chlamydia trachomatis (11) and HIV-1 (12), or why gonococci significantly increases viral shedding by HIV-1-infected individuals (12,13), is still uncertain. However, collectively, these observations are consistent with N. gonorrhoeae being able to directly influence the immune response. The mechanisms determining such effects have yet to be elucidated.

The neisserial colony opacity-associated (Opa) proteins govern bacterial adhesion to, and uptake into, host cells (14). A single strain of N. gonorrhoeae encodes up to eleven different opa alleles and expression from each locus is phase variable, being turned on and off at a rate of ˜1 per 10³ cells/generation/locus. The natural ligands of most Opa variants have been well defined. Some variants, typified by the Opa₅₀ variant of gonococcal strain MS11, bind to heparan sulfate proteoglycans (HSPG), including cell surface-expressed syndecan receptors, and to the extracellular matrix proteins vitronectin and fibronectin (14). A second class of Opa variants, including the antigenically distinct, but functionally conserved, Opa₅₂ and Opa₅₇ variants of strain MS11, are specific for various members of the carcinoembryonic antigen-related cellular adhesion molecule (CEACAM; formerly CD66) receptor family. This is a highly specific, protein-protein interaction, which allows individual Opa variants to bind various combinations of CEACAM1, CEACAM3, CEACAM5 and/or CEACAM6 (14). While non-opaque gonococcal isolates can establish an infection following urethral challenge in human male volunteers, the bacteria recovered are predominantly Opa⁺ (15-17). Previous studies have attempted to relate size and/or immunological reactivity with clinical symptoms associated with individual gonococcal infections (4,18), however, neither of these characteristics correlate with the receptor specificity of individual Opa variants (19). However, ˜94% of a diverse set of gonococcal isolates obtained from mucosal infections bind CEACAM1 (20). Together, these studies suggest that the expression of CEACAM1-specific Opa phase variants is strongly favored in vivo.

CEACAM proteins are members of the immunoglobulin superfamily, and individual family members are differentially expressed on various tissues in vivo (21). CEACAM1, previously CD66a or biliary glycoproteins (BGP), is unique within this group as it contains an immunoreceptor tyrosine-based inhibitory motif (ITIM) in its cytoplasmic domain (21,22). The ITIM is present in various coinhibitory receptors that function to antagonize kinase-dependent signaling cascades initiated by lymphocyte activation (23). This inhibitory effect is triggered by the phosphorylation of tyrosine residues within the ITIM, which results in recruitment of the Src homology 2 (SH2) domain-containing tyrosine phosphatases such as SHP-1 (24) and SHP-2 (25), and the SH2-containing inositol phosphatase SHIP (26). Consistent with these attributes, CEACAM1 associates with SHP-1²⁷ and SHP-2 (28) following pervanadate treatment of cells or in the presence of a constitutively active tyrosine kinase (27,28) and CEACAM1 recruitment of these phosphatases appears to mediate the receptor's ability to arrest tumor cell growth (29,30).

CEACAM1 is the only receptor of the CEACAM family that is expressed by human lymphocytes (31). The influence of this receptor on lymphocyte activation is, however, unclear. CEACAM1-specific antibodies have been reported to either enhance (31,32) or reduce (33) the activation of T lymphocytes in response to T cell receptor (TCR) cross-linking in vitro.

### SUMMARY OF THE INVENTION

The present inventors have demonstrated that ligating a member of the carcinoembryonic antigen (CEA) family, designated CEACAM1, with neisserial colony opacity-associated (Opa) proteins results in suppression of a T lymphocyte response. Accordingly, the invention provides a method of modulating, preferably suppressing, an immune response comprising administering an effective amount of a bacterial protein, preferably an Opa protein, to an animal or cell in need thereof.

The present inventors have also found that ligating CEACAM1 with Opa proteins inhibits the growth of a lymphocytic tumor cell line. Accordingly, the present invention also provides a method of inhibiting the growth of a tumor cell comprising administering an effective amount of a bacterial protein, preferably an Opa protein, to an animal or cell in need thereof.

The present invention also includes pharmaceutical compositions for use in modulating an immune response or in inhibiting tumor cell growth comprising an effective amount of a bacterial protein, preferably an Opa protein, in admixture with a suitable diluent or carrier.

Other features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

The invention will now be described in relation to the drawings in which:

FIGS. 1A-C are graphs showing the determination of CEACAM1 expression and surface exposure in primary CD4⁺ T lymphocytes. (a) CD4⁺ T cells isolated from human blood were either left unstimulated (upper panel) or were stimulated using IL-2 (lower panel). Monoclonal antibody D14HD11 was used to detect CEACAM1 expression on CD4⁺ T lymphocytes by flow cytometry. (b) Purified CD4⁺ T cells were stimulated with indicated concentrations of IL-2, and CEACAM1 expression determined by western blot analysis of cellular extracts using the CEACAM-specific monoclonal antibody D14HD11. (c) Purified CD4⁺ T cells maintained in the presence of no exogenous stimulus (Unstimulated), anti-CD3ε monoclonal antibody (a-CD3), or a mixture of anti-CD3ε and anti-CD28 (α-CD3+α-CD28) monoclonal antibodies, as indicated. CEACAM1 expression was determined as in (b).

FIG. 2 are graphs showing the determination of CD69 surface expression by primary CD4⁺ T lymphocytes. Purified CD4⁺ T cells were either left unstimulated, stimulated using anti-CD3ε, or by using a combination of antibodies specific for CD3ε and CD28 (α-CD3+α-CD28), each in the presence of either nothing (a), Opa₅₀-expressing N. gonorrhoeae (b), Opa₅₂-expressing N. gonorrhoeae (c), control immunoglobulin (d), or CEACAM-specific immunoglobulin (e). The proportion of the CD4⁺ T cell population that expresses the CD69 early activation marker was determined by flow cytometry, after analysis of at least 5,000 cells from each sample. Data shown is from one experiment, and is representative of trends that were apparent in at least 3 independent experiments. When using data from independent experiments to test statistical significance, the probability that CD69 expression levels in response to Opa₅₂-expressing gonococci are the same as that observed in response to Opa₅₀-expressing gonococci is, in each condition, ≦0.015. The probability that α-CEA samples are the same as Ig(−) samples is, in each case, ≦0.009, except for the Ig(−) versus α-CEA samples with CD3 alone, where P=0.04.

FIG. 3 is a graph showing the effect of CEACAM1 ligation on IL-2-dependent proliferation of primary CD4⁺ T lymphocytes. (a) Influence of expressed adhesin on CD4⁺ T lymphocyte proliferation in response to N. gonorrhoeae. Purified CD4⁺ T cells were stimulated using IL-2 and monoclonal antibodies specific for CD3ε in the presence of N. gonorrhoeae expressing either no adhesin (Opa⁻), the HSPG-specific Opa₅₀, the CEACAM-specific Opa₅₂ or Opa₅₇, pilus (Opa⁻Pilus⁺) or no bacteria (−). Lymphocyte culture density was determined by direct counting using a hemocytometer following 144 h, with results being expressed as a percentage of the initial culture density. (b) Influence of bacterial density on IL-2-dependent CD4⁺ T lymphocyte proliferation. Purified CD4⁺ T cells were exposed to IL-2 in the presence of indicated densities of N. gonorrhoeae expressing either Opa₅₀ or Opa₅₂, with densities being expressed as bacteria per lymphocyte (Multiplicity of Infection, MOI). Lymphocyte culture densities, determined as described in (a), are indicated in the left panel. The Index of Proliferation, which indicates the relative increase in culture density in the presence of Opa₅₂-expressing gonococci as compared to the increase occurring in the presence of Opa₅₀-expressing bacteria, is displayed in the right panel. (c) Influence of CEACAM-specific (α-CEA) versus control (Ig(−)) immunoglobulin on IL-2-dependent proliferation of CD4⁺ T cells. Purified CD4⁺ T cells were exposed to IL-2 in the presence of indicated concentrations of antibody, and the culture density attained is displayed in the left panel. The Index of Proliferation, indicating the relative increase in lymphocyte culture density following exposure to anti-CEACAM versus control immunoglobulin is displayed in the right panel. (d) Influence of anti-CEACAM versus control immunoglobulin on lymphocyte proliferation in response to N. gonorrhoeae expressing Opa₅₀. Lymphocytes were exposed to IL-2, Opa₅₀-expressing gonococci and either anti-CEACAM or control antibodies, as indicated. Proliferation was determined as outlined in (a). In each case (a-d), data is representative of at least 2 independent experiments. Data presented are mean±standard deviation of ≧4 parallel samples.

FIG. 4 is a graph showing the influence of CEACAM1 ligation on the proliferation of primary CD4⁺ T lymphocytes. Purified CD4⁺ T cells were stimulated with IL-2, anti-CD3ε monoclonal antibody and/or anti-CD28 monoclonal antibody, each in the presence of Opa₅₀ or Opa₅₂-expressing N. gonorrhoeae, anti-CEACAM (a-CEA) or control (Ig(−)) immunoglobulin, or none of these (−), as indicated. Proliferation of purified CD4⁺ T lymphocyte cultures was determined as outlined in FIG. 3. In each case, data is representative of at least 3 independent experiments. Data presented are mean±standard deviation of ≧24 parallel samples.

FIG. 5 are graphs showing the characterization and quantification of cell death in primary CD4⁺ T lymphocytes. Purified CD4⁺ T cells were either left unstimulated (a) or were stimulated using a combination of anti-CD3ε and anti-CD28 (b), IL-2 (c), or a combination of IL-2 and anti-CD3ε and anti-CD28 (d), each together with Opa₅₀ or Opa₅₂-expressing N. gonorrhoeae, anti-CEACAM (α-CEA) or control (Ig(−)) immunoglobulin, or none of these (−), as indicated. Viability staining was done using annexin-V-FLUOS and propidium iodide, and live, dead, apoptotic and necrotic cellular populations were quantified by flow cytometry. Data presented is representative of three independent experiments.

FIG. 6 shows SHP-1 and SHP-2 tyrosine phosphatases associate with CEACAM1 that is bound by Opa₅₂-expressing N. gonorrhoeae. Following stimulation with indicated concentrations of anti-CD3ε, purified CD4⁺ T cells were infected with N. gonorrhoeae expressing either the HSPG-specific Opa₅₀ or CEACAM-specific Opa₅₂. Lymphocyte membranes were then solubilized, and gonococci recovered by differential centrifugation of the lysates. Component proteins in the bacteria-containing pellets were then analyzed by immunoblot analysis to detect CEACAM1 (a), SHP-1 (b), and SHP-2 (c) that remained associated with each N. gonorrhoeae strain.

FIG. 7 is a graph showing the Jurkat CD4⁺ T lymphocytes cell line express CEACAM1 in response to prestimulation with IL-2.

FIG. 8 is a scanning electron micrograph showing the transformed CD4⁺ Jurkat T cells with adherent gonococci [N313].

FIG. 9 is a graph showing the quantification of adherent gonococci recovered from Jurkat cells, as determined by saponin-mediated lysis of the Jurkat cell membranes and dilution plating of recovered lysates.

FIG. 10 is a graph showing the proliferation of Jurkat cells in response to gonococcal infection.

FIG. 11 is a graph showing Jurkat proliferation in response to infection with N313: live or killed bacterial challenge dose.

FIG. 12 is a graph showing Jurkat proliferation in response to anti-CEACAM serum or infection with N. gonorrhoeae expressing either the CEACAM-specific Opa₅₇ protein (strain N313) or pilus (strain N496). Gonococcal infection [+/−αCD3 and IL-2] was for 48 hours.

FIG. 13A are graphs showing Jurkat proliferation in response to anti-CEACAM1 serum [+/− anti-CD3 and IL-2] for 48 hours, as determined by ³H thymidine incorporation: anti-CEACAM antibody reduces the normal growth of the immortalized Jurkat cell line.

FIG. 13B are graphs showing Jurkat proliferation in the presence of anti-CEACAM1 serum [+/− anti-CD3 and IL-2]—48 hours—determined by ³H thymidine incorporation: activation of T cell receptor increases the ability of anti-CEACAM antibody to inhibit the proliferation of Jurkat cell.

FIG. 13C is a graph showing the % reduction in proliferation of Jurkat cells challenged with anti-CEACAM antibodies [50 μg/ml/48 h].

FIG. 14 is a graph showing the apoptosis and necrosis of Jurkat cells in response to gonococcal infection or challenge with anti-CEACAM1 antibodies.

FIG. 15 is a schematic showing a CD4⁺ T lymphocyte inhibition model.

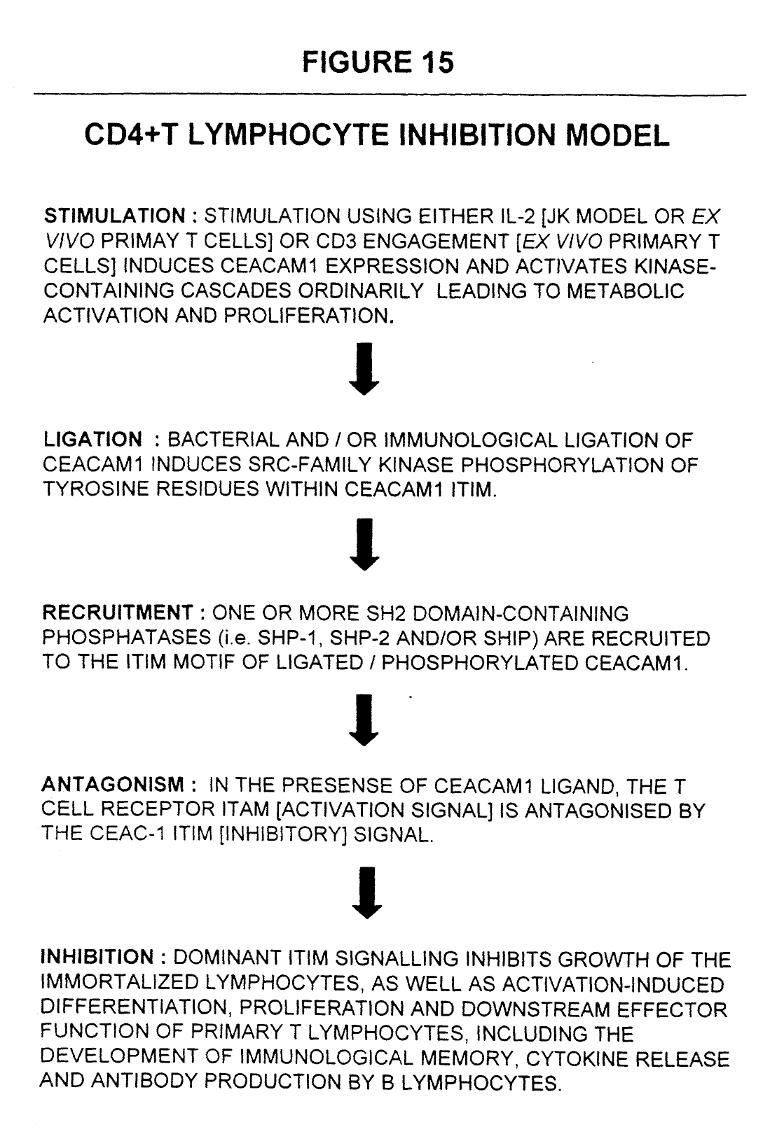

FIG. 16A (and SEQ ID NOs:1-10) shows oligonucleotide primers used to amplify single loops from Neisseria gonorrhoeae MS11 opa variants.

FIG. 16B is a schematic representation of predicted two-dimensional structure of neisserial Opa proteins. Figure adapted from Bhat et al. (1993) Molecular Microbiology 5:1889-1901. Arrowheads delineate predicted borders of Opa fragments encoded by gene fragments amplified using corresponding oligonucleotides shown in (A).

FIG. 16C shows PCR product obtained with oligonucleotide primers listed in FIG. 16A when using opa51 allele as template DNA.

FIG. 17A (and SEQ ID NOs:11 and 12) shows the sequence of the pG8ASET phagemid vector. Region encoding recombinant M13 filamentous phage gene VIII protein is shown, indicating gene VIII nucleotide and protein sequences, Ncol cloning site used to insert opa fragments, PG8-F and PG8-R primer binding sites, suppressible stop codon (*), and sequence of E-tag epitope expressed when inserted fragment shifts the translational reading frame to allow translation of downstream gene VIII residues. Figure taken from Dareyl Vaz (2000) Analysis of factors contributing to colonization of epidemic Canadian multi-drug resistant Staphylococcus aureus strain CMRSA-1. MSc thesis submitted to Graduate Department of Laboratory Medicine and Pathobiology, University of Toronto.

FIG. 17B (and SEQ ID NOs:13 and 14) shows the oligonucleotide primer sequences used to detect insertion of fragments cloned into Ncol cloning site.

FIGS. 18A and B are graphs showing CEACAM-specific binding of recombinant phage expressing Opa-derived peptides. Number of ampicillin-resistant colonies (A) and E-tag expressing (B) colonies recovered following panning over HeLa-CEACAM cells. Helper phage and uninfected TG-1 bacterial controls were used to define background threshold recovery of antibiotic resistant and/or E-tag expressing colonies.

### DETAILED DESCRIPTION OF THE INVENTION

I. Immune Modulation

The inventors have demonstrated that ligation of the CEACAM1 receptor on primary lymphocytes isolated from peripheral human blood suppresses the response of these cells to stimuli that is otherwise activating. Specifically, ligation of CEACAM1 by Neisseria gonorrhoeae expressing the Opa₅₂ protein reduces the lymphocytes' expression of CD69, an early marker of lymphocyte activation. This treatment also reduces lymphocyte proliferation in response to the activating cytokine IL-2 and/or stimulation through ligation of the CD3-epsilon component of the T cell receptor, either alone or together with the co-stimulatory receptor CD28. This effect occurs in a dose-dependent manner, and appears to be due to an inhibition of activation rather than CEACAM1-mediated toxicity to the cells, since no significant increase in cell death occur in response to Opa₅₂-expressing bacteria as compared to appropriate controls.

Accordingly, the present invention provides a method of modulating an immune response comprising administering an effective amount of a bacterial protein to an animal or cell in need thereof.

The term “immune response” as used herein includes any response of the immune system including both cell mediated and humoral responses.

The term “modulating an immune response” as used herein includes both upregulation or activation of an immune response as well as downregulation or suppression of an immune response. In a preferred embodiment, the immune response is suppressed.

The term “suppressing an immune response” as used herein means that the immune response in the presence of the bacterial protein is reduced, lowered or inhibited as compared to the immune response in the absence of the bacterial protein.

The term “effective amount” as used herein means an amount effective, at dosages and for periods of time necessary to achieve the desired result (e.g. to suppress an immune response). The effective amount of a compound of the invention may vary according to factors such as the disease state, age, sex, and weight of the animal. Dosage regima may be adjusted to provide the optimum therapeutic response. For example, several divided doses may be administered daily or the dose may be proportionally reduced as indicated by the exigencies of the therapeutic situation.

The term “animal” as used herein includes all members of the animal kingdom, including humans. Preferably, the animal to be treated is a human.

The term “a cell” as used herein includes a single cell as well as a plurality or population of cells. Administering an agent to a cell includes both in vitro and in vivo administrations. The cell can be any cell that expresses CEACAM1 including, but not limited to, T lymphocytes, B lymphocytes, NK cells, monocytes, macrophage, granulocytes and dendritic cells. The T lymphocytes can be any type including CD4⁺ T cells, CD8⁺ T cells, double negative (CD4⁻CD8⁻) T cells, double positive (CD4⁺ CD8⁺) T cells and γδT cells. In a specific embodiment, the T lymphocytes are CD4⁺ helper T lymphocytes, e.g. lymphocytes that produce cytokines in order to modulate the activity of other immune cells.

The term “administering a bacterial protein” as used herein includes both the administration of a bacterial protein as well as the administration of a nucleic acid sequence encoding a bacterial protein. In the latter case the bacterial protein is produced in vivo.

The term “bacterial protein” as used herein means a protein that is derived from a bacteria that is useful in modulating an immune response. Preferably, the bacterial protein is a CEACAM1 binding protein. Most preferably, the bacterial peptide is obtained from Neisseria sp. or Haemophilus sp.

In a preferred embodiment, the bacterial protein is an Opa protein.

The term “Opa protein” as used herein means a neisserial opacity associated protein or a fragment, analog, derivative, variant or mimetic of any Opa protein that can modulate or suppress an immune response or inhibit tumor growth. Preferably, the Opa protein can bind to CEACAM1 and cause ligation of CEACAM1 with consequent immune suppression or inhibition of an immune cell.

Opa proteins that may be used in the present invention include, but are not limited to, all of the Opa proteins listed in Table 1, as well as variants, analogs, derivatives of mimetics of any of these. Specific Opa proteins that may be used include Opa₅₂ and Opa₅₇. Since the neisserial Opa proteins are highly antigenically variable, the Opa protein may be any of the Opa protein variants that can be expressed by various neisserial species and that also bind to the CEACAM1 receptor. Opa proteins include the Opa proteins encoded by any neisserial species, including the pathogenic Neisseria gonorrhoeae and Neisseria meningitidis and the commensal species such as Neisseria lactamica and Neisseria subflava, for which their Opa proteins have been shown to bind CEACAM1 (78), and other commensals that also express Opa proteins.

The term “Opa protein” also refers to analogous proteins from other bacterial species. This includes, but is not restricted to, the CEACAM1-binding proteins of Haemophilus influenzae. Like the neisserial Opa proteins, the H. influenzae P5 proteins are antigenically variable outer membrane proteins that are predicted to form a beta-barrel structure with eight transmembrane regions and four extracellular loops. As with the Opa proteins, the P5 transmembrane regions and the 4th surface-exposed loop are well conserved, while the sequence within the other surface-exposed loops is variable (79,80). Also like various of the neisserial Opa proteins, the H. influenzae P5 proteins function in attachment to host cells via binding to CEACAM receptors, including CEACAM1 (81). This interaction parallels that seen with the neisserial Opa proteins in that P5 binding to CEACAM is a protein-protein interaction and similar point mutations within the host receptor abrogate binding to both of these bacterial adhesins (82). Therefore it is expected that P5-mediated ligation of CEACAM1 will have the same immunosuppressive effect as do the CEACAM1-binding Opa proteins.

The term “Opa protein” also includes variants, analogs, derivatives, mimetics or fragments of an Opa protein.

The term “analog” as used herein includes any peptide having an amino acid residue sequence substantially identical to any of the known Opa sequences in which one or more residues have been conservatively substituted with a functionally similar residue and which displays the ability to bind CEACAM1. Examples of conservative substitutions include the substitution of one non-polar (hydrophobic) residue such as alanine, isoleucine, valine, leucine or methionine for another, the substitution of one polar (hydrophilic) residue for another such as between arginine and lysine, between glutamine and asparagine, between glycine and serine, the substitution of one basic residue such as lysine, arginine or histidine for another, or the substitution of one acidic residue, such as aspartic acid or glutamic acid for another. The phrase “conservative substitution” also includes the use of a chemically derivatized residue in place of a non-derivatized residue provided that such polypeptide displays the requisite activity.

The term “derivative” as used herein refers to a peptide having one or more residues chemically derivatized by reaction of a functional side group. Such derivatized molecules include for example, those molecules in which free amino groups have been derivatized to form amine hydrochlorides, p-toluene sulfonyl groups, carbobenzoxy groups, t-butyloxycarbonyl groups, chloroacetyl groups or formyl groups. Free carboxyl groups may be derivatized to form salts, methyl and ethyl esters or other types of esters or hydrazides. Free hydroxyl groups may be derivatized to form O-acyl or O-alkyl derivatives. The imidazole nitrogen of histidine may be derivatized to form N-im-benzylhistidine. Also included as derivatives are those peptides which contain one or more naturally occurring amino acid derivatives of the twenty standard amino acids. For examples: 4-hydroxyproline may be substituted for proline; 5 hydroxylysine may be substituted for lysine; 3-methylhistidine may be substituted for histidine; homoserine may be substituted for serine; and ornithine may be substituted for lysine. Polypeptides of the present invention also include any polypeptide having one or more additions and/or deletions or residues relative to the sequence of an Opa protein, so long as the requisite activity is maintained.

The term “peptide mimetic” as used herein includes synthetic structures which may or may not contain amino acids and/or peptide bonds but retain the structural and functional features of an.Opa protein or peptide. Peptide mimetics also include peptoids, oligopeptoids (83); and peptide libraries containing peptides of a designed length representing all possible sequences of amino acids corresponding to an Opa protein. Peptide mimetics may be designed based on information obtained by systematic replacement of L-amino acids by D-amino acids, replacement of side chains with groups having different electronic properties, and by systematic replacement of peptide bonds with amide bond replacements. Local conformational constraints can also be introduced to determine conformational requirements for activity of a candidate peptide mimetic. The mimetics may include isosteric amide bonds, or D-amino acids to stabilize or promote reverse turn conformations and to help stabilize the molecule. Cyclic amino acid analogues may be used to constrain amino acid residues to particular conformational states. The mimetics can also include mimics of inhibitor peptide secondary structures. These structures can model the 3-dimensional orientation of amino acid residues into the known secondary conformations of proteins. Peptoids may also be used which are oligomers of N-substituted amino acids and can be used as motifs for the generation of chemically diverse libraries of novel molecules.

The term “fragment” refers to any subject peptide having an amino acid residue sequence shorter than that of an Opa protein (including analogs, derivatives or mimetics) that retains the ability to modulate or suppress an immune response or inhibit tumor growth.

The results in Example 3 show that loops 2 and 3 of several Opa variants can bind to CEACAM1. These loops contain hypervariable regions. Accordingly, the present invention includes the use of Opa proteins comprising all or part of a hypervariable region, preferably loop 2 or loop 3, which contain hypervariable regions 1 (HV1) and 2 (HV2), respectively, from an Opa protein.

One skilled in the art will appreciate that many assays can be used to determine if a particular Opa protein or fragment thereof is useful in the methods of the invention. In particular, functional Opa peptides, e.g. those capable of binding to CEACAM1, or capable of inducing immunomodulation in the target (effector) cell, or of inhibiting homotypic and/or heterotypic interactions between CEACAM1 and other potentially interacting proteins could be identified and otherwise evaluated using any or all of the following techniques.

Random or specifically cloned genomic fragments of Opa derived from commensal or pathogenic Neisseria sp., Haemophilus influenzae, or other bacterial species known to bind CEACAM receptors could be prepared as recombinant fragment(s) to be expressed from a plasmid or other suitable expression vector using standard molecular biology techniques (75). Nucleotide sequences derived from the Neisseria gonorrhoeae and/or N. meningitidis genomes (i.e. genomic sequences including Opa coding sequences) could be prepared as a plasmid or, if appropriate, a cosmid library using standard molecular biology techniques (75). Such fragments may be expressed as isolated peptides or may be peptides fused to carrier proteins, including those such as maltose binding protein, cellulose binding protein, green fluorescent protein (GFP) (or a similar fluorescent or luminescent protein), hexahistidine, or the Fc (or other) fragment of immunoglobulin. In some cases, mutation or insertion of sequences adjacent to the cloned fragments may be performed to introduce novel residues or amino acid sequences which may influence the surface expression and/or folding of the inserted peptide, and/or otherwise influence the ability of this fragment to bind CEACAM1. In addition to their influence on topology and/or function of the cloned fragments, such modification may also be used to simplify purification of the recombinant proteins by using standard biochemical techniques (i.e. affinity or immunoaffinity chromatography), or may allow the more efficient determination of protein expression and/or localization of the recombinant peptide (i.e. using semi-quantitative immunoblotting probed using antibody(ies) directed against the novel peptide sequence).

Genes or gene fragments encoding recombinant peptides or fusion proteins derived from a CEACAM-binding organism (e.g. Opa genes from Neisseria sp.) may be expressed in the context of recombinant bacteriophage (i.e. rM13), or, alternatively, in the context of a bacterial cell (i.e. Escherishia coli BL21 de3) transformed using established molecular biology techniques or, if deemed appropriate, in the context of a suitably transfected mammalian, insect or other cell line (75) for the purpose of expressing the recombinant peptide or proteins. Such gene transfer may be enabled using established molecular biology techniques including those involving retroviral, liposome mediated or DEAE dextran mediated transfection, or similar techniques.

Protein expression, stability and localization could be assessed using sodium dodecyl sulfate polyacrylamide gel electrophoresis, immunoblots (probed using antibody(ies) directed against the expressed peptides, fluorescence activated cell sorting (in which the “target” protein is fluorescently labeled (either directly or otherwise), or immunofluorescent microscopy (or similar techniques).

As an alternative to expressing cloned nucleotide fragments from bacteria or viruses known to bind CEACAM receptors, synthetic peptides may be synthesized based upon known sequence of the CEACAM-binding adhesin(s). Subfragments of the adhesin may also be generated by enzymatic or chemical cleavage of such proteins.

Peptides expressed in any or all of these manners could be assessed for functionality (e.g. capable of binding CEACAM1 or capable of modulating an immune response) using any or all of the following techniques:

One potential binding partner, being either CEACAM1 (or a derivative of this receptor) or the test peptides or preparation including such moieties, could be immobilized on a nitrocellulose membrane (or other solid support) and thereafter incubated with the other putative binding partner prepared in a soluble form. The soluble binding partner may be labeled using either chemical, radiological, luminescent, fluorescent or other label, either covalently coupled or indirectly bound (e.g. via a labeled antibody). Colorometric, fluorometric, chemiluminescent and/or radiochemical techniques can then be used, as appropriate, to allow detection of the soluble partner that is bound to the insoluble component. Alternatively, a competition reaction could be used, in which the soluble test factor may be left unlabelled and then mixed with a known CEACAM1 binding partner that is labeled, allowing the binding of the test component to be detected by its ability to compete off binding of the labeled substrate. In this and related techniques, the immobilized binding partner could be either the recombinant peptide or the putative ligand thereto. Detection methodology would be altered as appropriate.

In a similar manner, interactions between recombinant peptide(s) and CEACAM or a CEACAM derived species could be determined using enzyme linked immunosorbant assay (ELISA). Briefly, one of the putative binding partners could be immobilized in a series of microwells, and incubated with a putative ligand or combination of differentially labeled known and putative ligands (i.e. a competition binding assay). Associated peptides could then be detected either by virtue of their intrinsic labels or by immunoreactivity with an antibody or antibodies or by affinity with a similar or other species, as described above. This process could be adapted to accommodate larger scale experiments by immobilizing one binding partner, i.e. CEACAM or a CEACAM derived species on agarose beads, sepharose, carboxymethyl dextran polymers, polyethylene glycol or other solid support, and allowing interaction with soluble recombinant peptides, either in a purified form or expressed in the any or all of the contexts described above. In procedures of this type there exist several chemical means of engineering immobilization, including, electrostatic pre-concentration on a plastic surface, covalent coupling using amine linkage, thiol coupling, or coupling by virtue of biotinylation and subsequent association with avidin. Such coupling reactions can be engineered using currently available proprietary reagents.

Furthermore, ligand binding, receptor stoichiometry and biomolecular affinity (as delineated by the association and dissociation constants ka and kd respectively) could be determined and quantified in real-time, and critically, in the fluid phase using surface plasmon resonance biosensor (BIAcoreX, BIAcore2000, or similar) hereinafter referred to as SPR. In this instance, CEACAM or a CEACAM-derived species could be immobilized using any or all of the techniques described above, and thereafter, putative ligands (i.e. recombinant peptides or combination of differentially labeled known and putative ligands) could be injected over the prepared surface, thereby allowing association. Such interactions can be monitored by virtue of alteration in the surface plasmon resonance angle (a quantitative optical effect displayed as a linear trace or “sensorgram” proportional to the quantity/weight of associated protein). This technique has also been used to determine receptor ligand stoichiometry, affinity of binding and reciprocal inhibition of ligand binding, by exposure of one binding partner (i.e. CEACAM) to a species other than its natural ligand or ligands.

Peptides, or species derived therefrom, may also be functionally assessed by panning over cell lines (viable or otherwise), which either express CEACAM receptors or can be induced, transformed or transfected in order to facilitate expression of such proteins or species. In such instances, non-specific association may be minimized by adsorbing putative ligands against cell lines which do not express the receptor(s) in question, and further by extensive washing following the initial association phase.

In each instance, novel ligands may be amplified either in the context of propagated recombinant phage (using established molecular biology techniques) or otherwise by propagation of the appropriate plasmid or cosmid, and subsequently engineered protein expression therefrom, in any or all of the contexts described above. Furthermore, the genetic sequences encoding novel ligands could be determined using established DNA sequencing techniques.

Finally, a peptide or peptides identified as novel ligands could be used as an experimental antigen either in the presence or absence of a carrier molecule, additional fusion peptide, and chemical adjuvant or as expressed in the context of a recombinant phage. Reactive serum or purified antibody or antibodies derived therefrom could be used in subsequent evaluation of ligands, for example by determining its ability to inhibit CEACAM receptor binding by bacteria or virus from which the peptide was derived, and as a tool for more general analyses involving any or all of the putative ligands and receptors either described or alluded to herein.

Accordingly, the invention provides a method of identifying Opa proteins which can bind with CEACAM1, comprising the steps of:

(a) reacting CEACAM1 and an Opa protein, under conditions which allow for formation of a complex between the CEACAM1 and the Opa protein, and

(b) assaying for complexes of CEACAM1 and the Opa protein, for free Opa protein or for non complexed CEACAM1, wherein the presence of complexes indicates that the Opa protein is capable of binding CEACAM1.

Conditions which permit the formation of Opa protein and CEACAM1 complexes may be selected having regard to factors such as the nature and amounts of the Opa protein and the protein.

The Opa protein-protein complex, free Opa protein or non-complexed proteins may be isolated by conventional isolation techniques, for example, salting out, chromatography, electrophoresis, gel filtration, fractionation, absorption, polyacrylamide gel electrophoresis, agglutination, or combinations thereof. To facilitate the assay of the components, antibody against CEACAM1 or the Opa protein, or labelled CEACAM1, or a labelled Opa protein may be utilized. The antibodies, proteins, or Opa proteins may be labelled with a detectable Opa protein as described above.

CEACAM1, or the Opa protein used in the method of the invention may be insolubilized. For example, CEACAM1 or Opa protein may be bound to a suitable carrier. Examples of suitable carriers are agarose, cellulose, dextran, Sephadex, Sepharose, carboxymethyl cellulose polystyrene, filter paper, ion-exchange resin, plastic film, plastic tube, glass beads, polyamine-methyl vinyl-ether-maleic acid copolymer, amino acid copolymer, ethylene-maleic acid copolymer, nylon, silk, etc. The carrier may be in the shape of, for example, a tube, test plate, beads, disc, sphere etc. The proteins or substance may also be expressed on the surface of a cell.

The insolubilized protein or Opa protein may be prepared by reacting the material with a suitable insoluble carrier using known chemical or physical methods, for example, cyanogen bromide coupling.

Once it is determined that an Opa protein binds to CEACAM1, one skilled in the art can determine whether or not it is useful in modulating such as suppressing an immune response. Immunosuppression could be determined, assessed, characterized and otherwise quantified in a variety of ways, including, but not restricted to, quantification of cell proliferation, either directly (i.e. by microscopic observation) or indirectly by incorporation of a radioisotope or otherwise labeled biosynthetic pre-cursor, nucleotide analogue and/or other species, i.e. tritiated thymidine, incorporation of bromodeoxyuridine (BRDU) or a similar molecule or by specific incorporation of a coloromentric dye (i.e a tetrazolium derivative or similar chemical species). Furthermore, functional characterization may be used as an indicator of immunosuppression and further, as a predictor of effects comensurate with any such response. Briefly, T lymphocytes, and more specifically those designated as “helper” T cells, secrete an array of cytokines into the extracellular milieu, and in so doing influence the function and activity of other cells, including those of the immune system (i.e. other lymphocytes, macrophages, neutrophils and natural killer cells) and, to some extent, cells of non-lymphoid lineage. Consequently, perturbation of cytokine expressions and/or secretion and/or receptor expression by any or all of these cell type may be indicative of immune modulation. Such effects can be characterized using a number of well established techniques, including enzyme linked immunosorbant assays (ELISA), by which cytokine and/or immunoglobulin secretion can be quantified in comparison to a pre-defined standard curve. In addition, cytokine dependent cell lines can be used to estimate cytokine secretion by any or all of the cell types described above. Among such cytokine dependent cell lines, viability varies directly as a function of exogenous cytokine levels (i.e. an interleukin 2 dependent cell line would proliferate and insodoing incorporate a marker molecule, only in the presence of exogenous IL-2). As such, supplementation of tissue culture fluid with supernatant from challenged and/or stimulated “test” cultures influences the survival of cytokine dependent cells—thereby establishing the cytokine content in the ‘test” supernatant. Cytokine expression can also be determined by intracellular cytokine staining in the context of an intact cell (or population thereof) in which the golgi apparatus has been disabled by treatment with brefeldin A (or a similar or analogous agent). Subsequent analysis by flow cytometry enables comparison of fluorescence intensity which is, in this instance, proportional to cytokine expression.

Immunosuppression might otherwise be determined by analysis of the rate and/or efficiency of antigen processing and presentation by antigen presenting cells (APC), and/or other cell types, and subsequent interactions with either the major histocompatibility complexes (MHC) I and/or II, and thereafter with the T cell receptor complex or subcomponents thereof or distinct receptor moiety. Furthermore, immunosuppressive effects influenced, at least in part, by ligation of cell surface receptors of the CEACAM and/or other families, may perturb the formation and/or maintenance and/or functional characteristics of the “immunological synapse” (IS) or focal contact point between the APC(s) and lymphocyte/lymphocytes. Disruption/modulation or other perturbation of this transient association, either in terms of its temporal duration or biochemical and/or biophysical composition, could be indicative of altered immune function. For example, co-association of CEACAM1 and the tyrosine phosphatases SHP-1 and or SHP-2 (and/or other phosphatase enzymes, or kinase enzymes and/or as yet uncharacterized species) either within, associated with, proximal to, or, in contrast, excluded from the IS, and coincident or subsequent variability in the phosphorylation of TCR receptor components and/or other ITAM/ITIM or similar motifs either directly or indirectly, would be suggestive of altered T cell function. Finally, either gross or localized variation in cellular tyrosine or other phosphorylation (as determined by flow cytometric techniques, ELISA based techniques, immunoblotting and/or other techniques), might be considered indicative of modulated immune function.

In a particular example, one can test the ability of an Opa protein to inhibit the proliferation of T lymphocytes exposed to stimulatory signals such as IL-2 and/or antibodies to the T cell receptor as described in detail in Example 1. Other immune assays that may be used, include assessing the ability to inhibit a cytotoxic T cell response, inhibit a mixed leucocyte reaction, inhibit antibody production, inhibit the production of a Th1 or Th2 cytokine, or increase the secretion of an immuneosuppressive cytokine. One can also test an Opa protein for its ability to suppress an immune response in vivo or for its ability to ameliorate a disease or condition where immune suppression is helpful.

There are many conditions or diseases in which immune suppression is a useful therapy including, but not limited to, preventing or treating autoimmune diseases, preventing or treating graft rejection, preventing or treating allergies, preventing or treating fetal loss as well as preventing or treating any inflammatory condition that is characterized by T cell proliferation and spontaneous activation.

A number of highly significant auto-immune disorders are characterized by excessive T cell activation and proliferation. These include host-graft intolerance, multiple sclerosis, rheumatoid arthritis, inflammatory bowel disease and insulin dependent diabetes mellitus. Furthermore, arteriosclerosis is also associated with T cell recruitment and localized aberrant inflammation. Recurrent spontaneous abortion is analogous to host/graft intolerance in which T lymphocyte subsets are aberrantly activated, resulting in fetal rejection. Clearly, controlled, non-toxic and specific suppression of T cell activation could have a significant therapeutic impact on each of these conditions.

Accordingly, the present invention provides a method of treating a disease or condition wherein it is desirable to suppress an immune response comprising administering an effective amount of a bacterial protein, such as an Opa protein, to an animal in need thereof.

As used herein, and as well understood in the art, “treating” is an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable. “Treating” can also mean prolonging survival as compared to expected survival if not receiving treatment.

The inventors suggest that, for the treatment of localized mucosal reactions, a suitable Opa protein could be presented on a non-absorbable particle or substrate (i.e. a micro-bead or gel). Fewer difficulties may be experienced using this method than in the treatment of systemic disease, where an entire cell population may be targeted. However, cell type and activation state can be characterized, in part, by receptor expression profiles (79). Consequently it may be possible to select a specific population dependent upon immunological reactivity.

II Tumor Inhibition

The inventors have also demonstrated that ligation of CEACAM1 using Neisseria gonorrhoeae expressing OPa₅₂ protein inhibits the growth of an immortalized lymphocyte cell line (Jurkat cells) following stimulation of these cells using IL-2 but in the absence of other activating or inhibitory stimuli. As with the immune-activated primary cells described above, these effects occur in a dose-dependent manner, and appear to be due to an inhibition of proliferation signals rather than CEACAM 1-mediated toxicity to the cells, since no significant increase in cell death occur in response to the Opa₅₂-expressing bacteria as compared to appropriate controls.

Accordingly, the present invention provides a method of preventing or inhibiting the growth of a tumor cell comprising administering an effective amount of a bacterial protein to an animal or cell in need thereof.

The term “preventing or inhibiting the growth of a tumor cell” means that the growth or proliferation of the tumor cell is reduced, lowered or inhibited as compared to the growth in the absence of the bacterial protein.

In a preferred embodiment, the bacterial protein is an Opa protein. The bacterial proteins and Opa proteins are described in Section I. The Opa proteins useful for tumor inhibition include Opa proteins (including fragments, analogs, derivatives and mimetics thereof) that are able to suppress tumor cell growth or proliferation. One of skill in the art can determine whether an Opa protein can prevent or inhibit tumor cell growth. For example, the assay described in Example 2 can be used.

The tumor cell can be any tumor that expresses or can be induced to express CEACAM1 and is preferably a lymphoma such as a T cell or B cell lymphoma.

The inventors' data indicate that proliferation of T lymphocytes is regulated by ligation of CEACAM1. IL-2, the pre-stimulating cytokine used in the analyses, is routinely employed as an adjunct to cancer chemotherapy, although the precise therapeutic mechanisms involved remain undefined (67). Our analyses confirm that IL-2 treatment induces expression of CEACAM1 in ex vivo purified CD4⁺ lymphocytes, and the inventors suggest that co-administration of IL-2 and an Opa protein may arrest aberrant growth of this population. In the treatment of cancer, IL-2 is frequently administered as an intravenous bolus, or in some cases by inhalation. Irrespective of administrative route, high does of IL-2 are typically associated with severe side effects (due, in part, to induction of IFNγ). The inventors speculate that combination therapy (as described above) may enable a reduction in IL-2 dose without compromising, and potentially improving, the overall efficacy of treatment. Furthermore, CEACAM1 expression by suitably stimulated tumor cells may enable the targeting of this population thereby negating the requirement for engineered therapeutic tropism. However, expression of specific cytokine receptors is a characteristic of certain tumor cells. Consequently, it may be possible to use these molecules in the targeting of malignant cells.

III. Pharmaceutical Compositions

The present invention also includes pharmaceutical compositions comprising an effective amount of one or more bacterial proteins, such as Opa proteins in admixture with a suitable diluent or carrier. Such compositions are useful in the above methods of immune suppression and cancer therapy.

In one embodiment, the invention provides a pharmaceutical composition for use in suppressing an immune response comprising an effective amount of a bacterial protein, such as an Opa protein in admixture with a suitable diluent or carrier.

In another embodiment, the invention provides a pharmaceutical composition for use in inhibiting tumor growth comprising an effective amount of a bacterial protein, such as an Opa protein in admixture with a suitable diluent or carrier.

The compositions described herein can be prepared by per se known methods for the preparation of pharmaceutically acceptable compositions which can be administered to subjects, such that an effective quantity of the active substance is combined in a mixture with a pharmaceutically acceptable vehicle. Suitable vehicles are described, for example, in Remington's Pharmaceutical Sciences (84) or Handbook of Pharmaceutical Additives (85). On this basis, the compositions include, albeit not exclusively, solutions of the substances in association with one or more pharmaceutically acceptable vehicles or diluents, and may be contained in buffered solutions with a suitable pH and/or be iso-osmotic with physiological fluids. In this regard, reference can be made to U.S. Pat. No. 5,843,456. As will also be appreciated by those skilled, administration of substances described herein may be by an inactive viral carrier.

The compositions may be administered in a convenient manner such as by injection (subcutaneous, intravenous, etc.), oral administration, inhalation, transdermal application, or rectal administration. Depending on the route of administration, the active substance may be coated in a material to protect the compound from the action of enzymes, acids and other natural conditions which may inactivate the compound.

The following non-limiting examples are illustrative of the present invention:

### EXAMPLES

### Example 1

Neisseria gonorrhoeae Opa Protein Binding to CEACAM1 (CD66a) Arrests the Activation and Proliferation of Human CD4⁺T Lymphocytes

In this Example, the inventors have demonstrated that gonococcal expression of Opa variants which bind to CEACAM1 inhibits the expression of the immediate early activation marker CD69 by primary CD4⁺ T lymphocytes that have been activated by cross-linking CD3, either alone or in association with the co-stimulatory receptor CD28. Furthermore, gonococci expressing the CEACAM-specific Opa₅₂ protein arrests the proliferation of CD4⁺ T lymphocytes in response to stimulation with IL-2 and/or ligation of the T cell receptor component CD3±CD28, as compared to that which occurred when these cells were either left uninfected or were infected with gonococci that express the Opa₅₀ variant which does not bind to CEACAM1. In each case, anti-CEACAM antibody mimicked the effect of Opa₅₂-expressing bacteria, indicating that CEACAM1 ligation alone was sufficient for the reduced T cell activation. Consistent with the receptor's ITIM being involved in this effect, CEACAM1 bound by Opa₅₂-expressing bacteria was found to be associated with the SH2-containing tyrosine phosphatases SHP-1 and SHP-2. Consequently, the inventors propose that gonococcal binding to CEACAM1 expressed by CD4⁺ T lymphocytes leads to the suppression of their normal response to activating stimuli. This diminished response would reduce the development of an effective immune response, both directly through a reduction in the number of effector T cells, and indirectly through the reduced activation of downstream effector cells. This is, to our knowledge, the first example of an immunosuppressive effect induced by bacterial ligation of an ITIM-containing co-inhibitory receptor.

### Materials and Methods

Bacterial strains

Gonococcal strains N302 (Opa⁻), N303 (Opa₅₀), N309 (Opa52), N313 (Opa₅₇) and N496 (Opa⁻Pilus⁺) which constitutively express single Opa variants or pilus (shown in parenthesis) have been described previously (58). These opa genes are expressed in a derivative of strain MS11 that has mutations abolishing the expression of the HSPG receptor-specific Opa₃₀. The ligands recognized by these various Opa variants have been described previously (14). Gonococci were grown from frozen stocks on GC agar (BBL™ Becton Dickinson Microbiology Systems, Cockeysville Md., USA) supplemented with 1% (v/v) lsoVitaleXTm enrichment (BBL™), and were sub-cultured daily, using a binocular microscope to monitor colony opacity phenotype. Opa expression and variant-type were routinely confirmed by SDS-PAGE (10%), with resolved proteins being transferred onto Immobilon P membrane (Millipore, Bedford Mass., USA) and probed using Opa cross-reactive monoclonal antibody 4B12/C 11 (59).

Purification of CD4⁺ T Lymphocytes

Lymphocytes were purified from citrated human peripheral blood using FICOLL PAQUE™ (Amersham Pharmacia Biotech, Baie d'Urfe, Quebec, Canada), according to the manufacturers specifications. CD4⁺ T lymphocytes were then isolated by negative selection using CELLECT™ PLUS purification columns (Cedar Lane Laboratories. Hornby, Ontario, Canada) according to the manufacturers specifications. Purified lymphocytes were routinely >85% CD3⁺CD4⁺ as determined by flow cytometry (data not shown), indicating an enrichment of this cell type and establishing the efficacy of this purification system. Cells were maintained in RPMI 1640 medium (Gibco-BRL, Burlington, Ontario, Canada) supplemented with 10% heat-inactivated fetal bovine serum (FBS; Gibco-BRL) at 37° C. in 5% CO₂ and humidified air.

Lymphocyte Stimulation

Where appropriate, isolated lymphocytes were stimulated using recombinant human IL-2 (1000 U/ml) (Pharmingen, Mississauga, Ontario, Canada) for 48 h prior to infection or antibody challenge. TCR stimulation was induced by treatment with 1 μg/ml mouse anti-human CD3ε IgG (clone UCHT1; Pharmingen), and, where indicated, costimulation was induced using 1 μg/ml mouse anti-human CD28 (clone CD28.2; Pharmingen). Stimulatory antibodies were cross-linked using 3 μg/ml sheep anti-mouse IgG -F(ab′)₂ (Sigma, Oakville, Ontario, Canada).

Flow Cytometric Analysis of Cell Surface Proteins

Lymphocyte purification efficiency was determined by quantification of CD3 and CD4 coexpression on purified lymphocytes. These surface proteins were detected using FITC-conjugated anti-CD4 antibodies (clone RPA-T4; Pharmingen, Mississauga, Ontario, Canada) and APC-conjugated anti-CD3 antibodies (clone UCHT1; Pharmingen). CD69 expression was detected using PE-conjugated anti-CD69 (clone FN50; Pharmingen), and CEACAM1 expression was detected using the monoclonal antibody D14HD11 (generously provided by Dr F. Grunert, University of Freiburg, Germany) followed by goat anti-mouse IgG conjugated to BODIPY-FL (Molecular Probes, Eugene, Oreg. USA). In each case 1-2×10⁶ lymphocytes were resuspended in 50 μl phosphate-buffered saline containing 1 mM MgCl₂ and 0.5 mM CaCl₂ (PBS/Mg/Ca) with 1% FBS and 0.05% sodium azide. Samples were then incubated with the appropriate antibodies, as indicated. A minimum of 5000 cells from each sample were then analyzed by flow cytometry using a FACSCalibur with CellQuest software (Becton Dickinson, San Diego, Calif., USA).

Lymphocyte Proliferation Assays

Purified CD4⁺ T lymphocytes were either stimulated with IL-2 (as described above) or left unstimulated. Lymphocytes were then prepared at a standardized cell density of 0.25-0.5×10⁶ cells/ml by direct counting using a Levy double hemocytometer. In some instances, additional immunological stimulation was induced via ligation of CD3ε, either alone or with co-ligation of CD28. These treatments were carried out simultaneously with the addition of bacteria infection or CEACAM-specific antibody. Infections and immunological treatments were carried out in RPMI+4 mM GlutaMAX (Gibco-BRL) supplemented with 5% (v/v) PBS/Mg/Ca and 1 U/ml benzonase endonuclease (Sigma), which was added to prevent gonococcal aggregation mediated by DNA released through bacterial autolysis (60). Multiplicity of infection (MOI) ranged from 0 to 200 bacteria/cell and, in parallel experiments, lymphocytes were challenged using either CEACAM-specific antibody solution (anti-CEA; DAKO Diagnostics, Mississauga, Ontario, Canada) or equal concentrations of non-reactive control antibody (DAKO Diagnostics) at concentrations ranging from 0-50 μg/ml. In some experiments lymphocytes were cochallenged with bacteria (MOI=200) and antibody (50 μg/ml). Gentamycin (50 μg/ml; Bioshop, Burlington, Ontario, Canada) was added to each sample 3 h after the commencement of infection and/or immunological challenge, and was maintained throughout the experimental time course to prevent gonococcal overgrowth during the extended proliferation experiments. In each case, lymphocyte density was assessed by direct counting using a hemocytometer at the commencement of the experiment and at indicated times post-infection/challenge. A standardized counting pattern was used throughout these analyses, and at least 12 quadrants were counted for each sample.

Characterization and Quantification of Cell Death

Purified CD4⁺ T lymphocytes were stimulated and either infected or immunologically challenged as described previously. At indicated times, cells were stained using Annexin-V-FLUOS/Propidium iodide (Boehringer Mannheim) according to the manufacturers specifications. Stained cells were analyzed by flow cytometry, allowing relative quantification of live, dead, apoptotic and necrotic populations.

Analysis of CEACAM1 Expression and Association with SHP-1 and SHP-2

In addition to analysis by flow cytometry (as described above), CEACAM1 expression was analyzed by SDS-PAGE (10%) and western blotting with the CEACAM-specific monoclonal antibody D14HD11. In several experiments, gonococci expressing either Opa₅₀ or Opa₅₂ were used to recover Opa-associated proteins from purified CD4⁺ T lymphocytes stimulated using either IL-2 (for 48 h) or 0-4 μg/ml anti-CD3ε IgG (for 3 h concurrent with infection) or, in separate experiments, 1 μg/ml anti-CD3ε IgG for 48, 96 or 144 h as indicated. Lymphocytes were infected at an MOI of 200 essentially as described previously, although in these experiments gentamycin treatment was omitted and cells were treated with cytochalasin D (1 μg/ml) for 30 min immediately prior to lysis to prevent cytoskeletal association of the receptors. Recovered cells were then lysed on ice using Tris buffer (50 mM, pH 7.4) containing 150 mM NaCl, 1 mM EDTA, 1% Triton X-100, 100 mM NaVO₄, 10 mM H₂O₂, 1 mM NaF, 1 mM PMSF, and 2 μg/ml each of aprotinin, leupeptin and pepstatin. After centrifugation at low speed, residual pellets, which include essentially intact N. gonorrhoeae (data not shown), were analyzed by SDS-PAGE (10% or 7.5%) and then western blotted with either monoclonal antibody D14DH11 or antiserum directed against either SHP-1 or SHP-2 (Santa Cruz Biotechnology).

Microscopic Analysis of Bacterial Binding and Uptake by Primary CD4⁺ T Lymphocytes

Lymphocytes were purified as described above and were either left unstimulated or stimulated with IL-2 as described above. These cells were then infected (MOI=10) using gonococcal strains pre-labelled with Texas Red^{R}-X, succinimidyl ester (Molecular Probes, Eugene Oreg.) according to the manufacturers specifications. Extracellular baceria were then labelled using the anti-gonococcal polyclonal serum (UTR01) which was raised against N. gonorrhoeae N302 (Opa⁻) using standard procedures. These bacteria were labeled using a BODIPY^{R}-FL conjugated secondary antibody (Molecular Probes, Eugene Oreg.). Intracellular versus extracellular bacteria were then distinguished by visualization using a Leica DM-IRBE inverted fluorescence microscope.

### Results

CEACAM1 Expression by Primary CD4⁺ T Lymphocytes Correlates with their Activation State

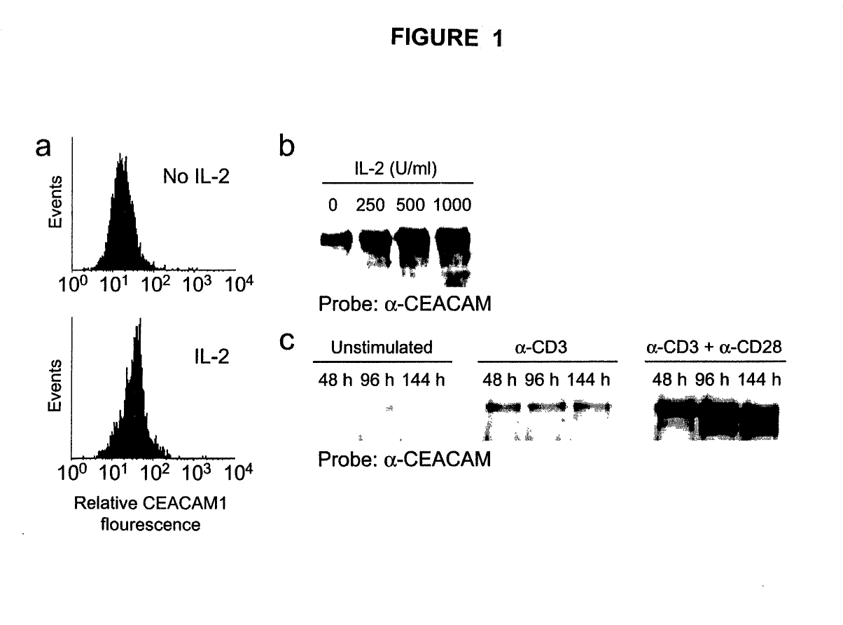

Lymphocyte expression of CEACAM1 correlates with their activation state (31-33). Consistent with this, increased expression of CEACAM1 was observed following interleukin-2 (IL-2) treatment of primary CD4⁺ T lymphocytes, both by flow cytometry (FIG. 1A) and immunoblot analysis (FIG. 1B). Receptor expression was stimulated in a dose-dependent manner by the addition of IL-2 (FIG. 1B). Ligation of CD3ε also induced CEACAM1 expression within 48 h, and no further increases in expression were detected after 96 or 144 h (FIG. 1C). Coligation of CD3ε and CD28 induced more CEACAM1 expression than was observed following ligation of CD3ε alone (FIG. 1C). In this case, induction of CEACAM1 expression was notable after 48 h and reached maximal and sustained amounts after 96 h (FIG. 1C).

CD69 Expression is Inhibited by CEACAM1 Ligation

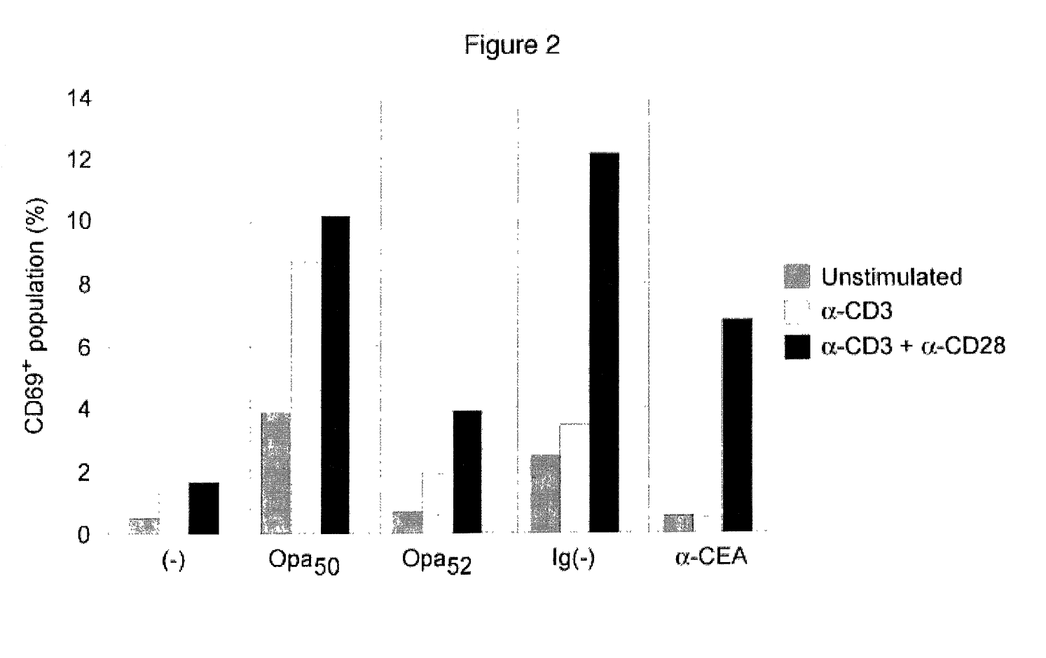

CD69 is a well characterized marker of lymphocyte activation, typically being expressed within 6-24 h following exposure to either mitogens or recall antigens (34,35). The inventors evaluated the effect of gonococcal infection and immunological challenge on lymphocyte expression of CD69 in response to various stimuli. In uninfected and unstimulated CD4+ lymphocytes, CD69 was expressed by <1% of the cell population (FIG. 2A). The number of CD69⁺ cells increased coincident with ligation of CD3ε, and further by coligation of CD3ε and CD28. However, in the absence of other stimuli, <2% of lymphocytes expressed CD69 in each of these conditions (FIG. 2A). Consistent with the reported influence of bacterial products, including lipopolysaccharides (35), on CD69 expression, infection with N. gonorrhoeae expressing the HSPG-specific Opa₅₀ protein increased the proportion of CD69⁺ cells to 3.9% -10.1% of the total population, depending on the method of stimulation (FIG. 2B). Comparable infection with an isogenic N. gonorrhoeae strain expressing the CEACAM-specific Opa₅₂ protein resulted in a much lower stimulatory effect. The influence of Opa₅₂ expression was most dramatic when cells were either left unstimulated or were stimulated by ligation of CD3ε alone. Under these conditions, cells infected with Opa₅₂-expressing gonococci were essentially indistinguishable from uninfected populations (compare FIGS. 2A-C, unstimulated and CD3). Following coligation of CD3ε and CD28 some lymphocyte stimulation was apparent, even in the presence of gonococci expressing Opa₅₂. However, the relative number of CD69⁺ cells was reduced by >60% in comparison to populations infected with gonococci expressing Opa₅₀ (compare FIGS. 2A-C, CD3-CD28).

In order to ascertain whether the difference between the gonococcal strains could result from an inhibitory effect of Opa₅₂ binding to CEACAM1 on T cell activation, the inventors tested the affect of CEACAM-specific antibodies on CD69 expression. Ligation of CEACAM1 with antibodies produced a similar result: CEACAM-specific antibodies inhibited lymphocyte activation in comparison to the control antibody (compare FIGS. 2D-E). CEACAM-specific antibody completely abrogated any increase in CD69 expression in response to CD3ε ligation, and reduced the number of CD69⁺ cells following coligation of CD3ε and CD28 by ˜45% (compare FIGS. 2D-E).

CEACAM1 Ligation Inhibits CD4⁺ T Lymphocyte Proliferation.

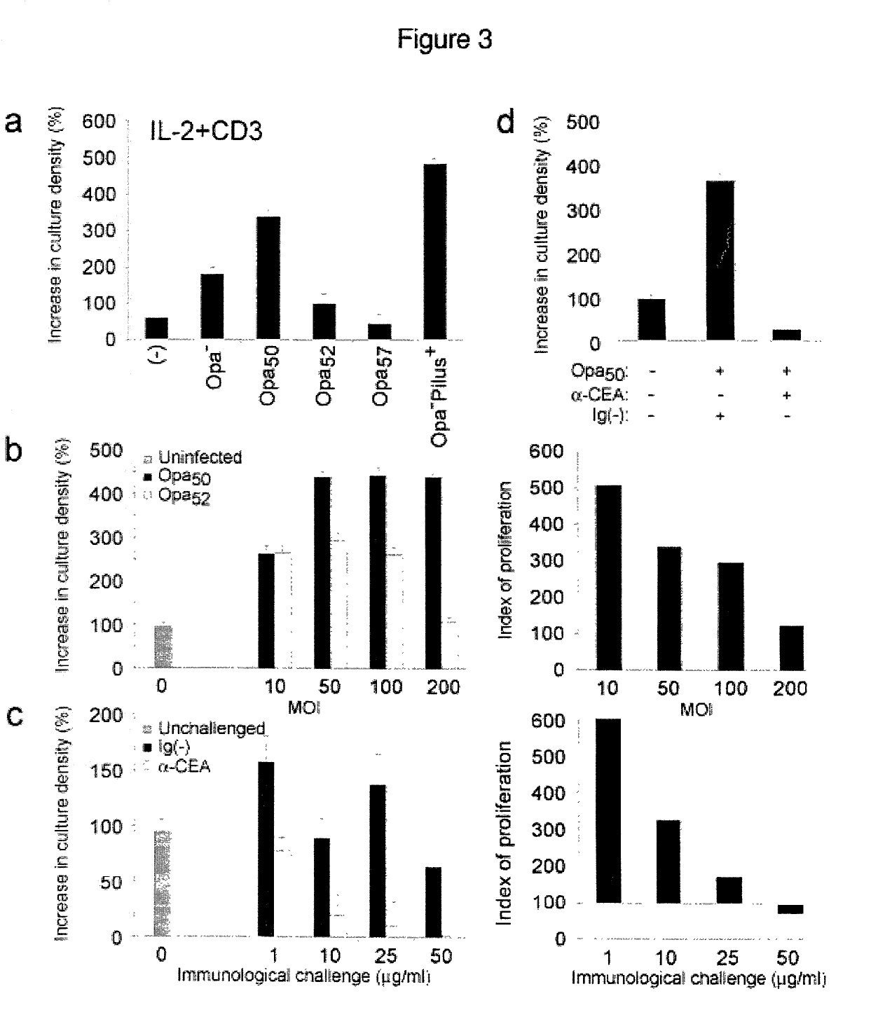

Subsequent to CD69 expression, clonal proliferation of activated CD4⁺ T lymphocytes results in an increased number of effector cells capable of propagating the immune response (34). Consequently, the inventors have investigated whether gonococcal infection also influenced CD4⁺ t lymphocyte proliferation in response to activating stimuli. Initially, lymphocytes stimulated with IL-2 and through ligation of CD3 were challenged using gonococci expressing either pilus, the HSPG-specific Opa₅₀, the CEACAM-specific Opa₅₂ or Opa₅₇ (14), or no adhesin. In experiments of this type, gonococci expressing the antigenically distinct, but functionally conserved, Opa₅₂ or Opa57 protein variants inhibited lymphocyte proliferation, whereas comparable challenge with other gonococcal strains stimulated lymphocyte proliferation relative to uninfected controls (FIG. 3A). Having established this trend, subsequent analyses employed only N. gonorrhoeae expressing Opa₅₀ as a control for Opa₅₂-expressing gonococci. The Opa₅₀-expressing strain was selected since Opa₅₀ is closely related to Opa₅₂, but binds to HSPG rather than CEACAM receptors, and yet our microscopic analyses established that these two strains were bound and internalized by primary CD4⁺ T cells at broadly comparable amounts (Opa₅₀: mean bacteria associated/lymphocyte=20, mean intracellular bacteria/lymphocyte=10 (50%); Opa₅₂: mean bacteria associated/lymphocyte=35, mean intracellular bacteria/lymphocyte=13 (37%)). Differences seen in the lymphocyte response were not, therefore, attributable to differences in bacteria association.

IL-2 treatment of the purified CD4⁺ T cells caused the population to double in size by 144 h (FIG. 3B, left panel). At a low multiplicity of infection (MOI=10), N. gonorrhoeae increased lymphocyte proliferation regardless of the Opa variant expressed. However, at MOI=50, infection with Opa₅₂-expressing gonococci reduced lymphocyte proliferation by 34% relative to infection using gonococci expressing Opa₅₀. At higher MOI a similar effect was noted, with Opa₅₂ reducing lymphocyte proliferation by 51% at MOI=100, and by 76% at MOI=200, essentially abrogating the stimulatory effect otherwise associated with gonococcal infection (FIG. 3B, left panel). Plotting the index of proliferation (proliferation in response to Opa₅₂/proliferation in response to Opa₅₀) clearly showed a dose-dependent inhibition of lymphocyte growth that correlated with gonococcal expression of Opa₅₂ (FIG. 3B, right panel).

In order to ascertain whether CEACAM1 ligation can itself influence the proliferation of primary CD4⁺ T lymphocytes, the inventors performed an immunological challenge using CEACAM-specific and control antibodies. Proliferation was typically lower in the absence of bacterial infection (FIG. 3C, left panel), however, CEACAM-specific antibody consistently reduced culture growth in comparison to treatment using control antibody. This effect was dose-dependent, with the CEACAM-specific antibody reducing proliferation by between 54 and 100%, depending upon the concentration used (FIG. 3C, left panel). Consistent with bacterial infections, plotting the index of proliferation in response to CEACAM-specific versus control antibody demonstrated the dose-dependent nature of this inhibitory effect (FIG. 3C, right panel).

To confirm that CEACAM1 ligation affected the stimulatory effect of gonococci on CD4⁺ T cells, the inventors exposed the lymphocytes to Opa₅₀-expressing N. gonorrhoeae in the presence of control or CEACAM-specific antibodies. Lymphocyte exposure to Opa₅₀-expressing bacteria and control antibody stimulated proliferation by 365% relative to uninfected cells. In contrast, cochallenge using this strain in combination with CEACAM-specific antibody instead reduced lymphocyte proliferation by ˜95% relative to that observed in the presence of control antibody (FIG. 3D). These results clearly showed that the observed differences in lymphocyte proliferation in response to Opa₅₀ versus Opa₅₂ were due to the latter's ability to ligate CEACAM1, and that this effect can overcome the stimulatory effect otherwise associated with Opa₅₀-expressing bacteria.

CEACAM1 Ligation Affects Response to Various Stimuli

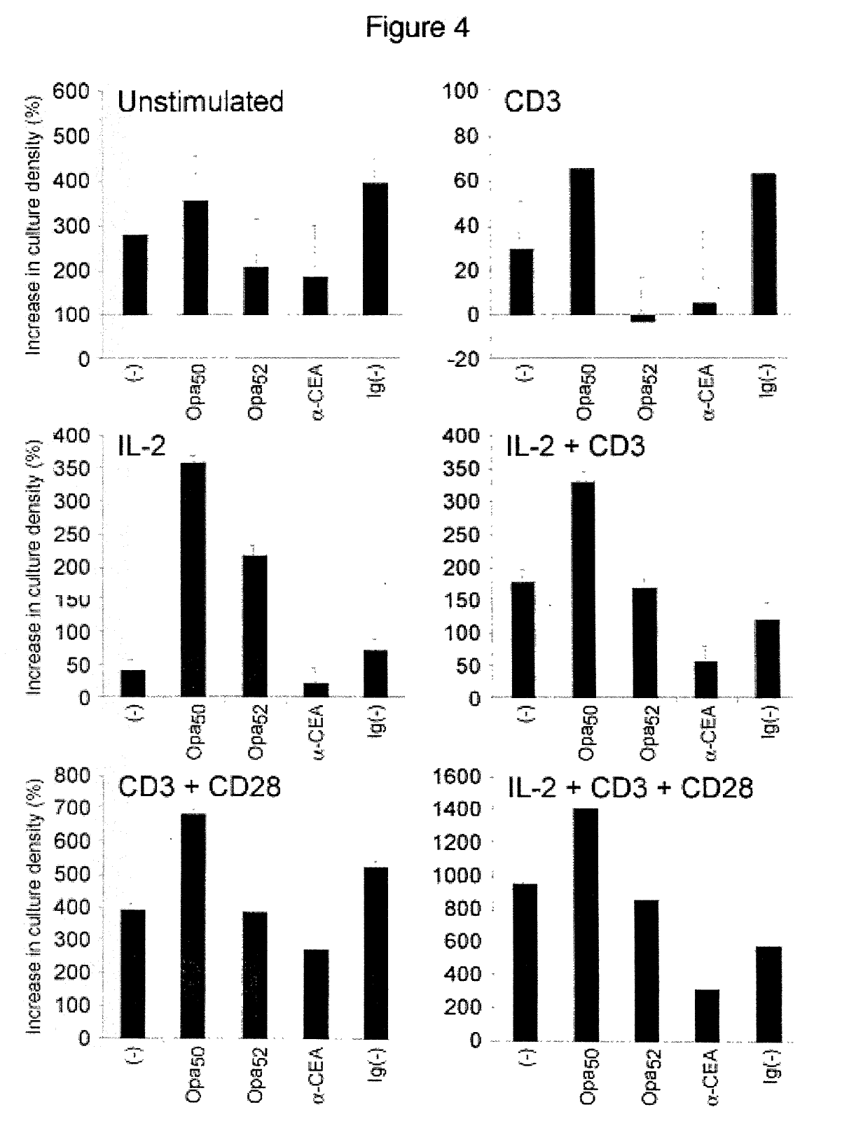

The inventors next sought to determine whether Opa-CEACAM1 interactions could suppress lymphocyte proliferation in response to other stimuli. Phosphorylation of tyrosine residues within an ITIM is required for the inhibitory function of coinhibitory receptors (36). Lymphocyte activation by the ligation of immunoreceptor tyrosine-based activation motif (ITAM)-containing receptors (i.e. the CD3ε component of the TCR) potently activates Src-family tyrosine kinases that can phosphorylate the ITIM of adjacent receptors (36). Subsequent recruitment and activation of phosphatases effectively increases the threshold of activating signals required to induce an effector response, and the relative strength of activating (ITAM) and antagonistic inhibitory (ITIM) signals determines the ultimate cellular response to stimulation. Consistent with such a model, the inventors observed that ligation of CEACAM1 by either N. gonorrhoeae Opa₅₂ or CEACAM-specific antibody had the most dramatic effect following coligation of the TCR (FIG. 4, CD3). Lymphocytes were exposed to various combinations of IL-2, CD3ε-specific antibodies, and CD28-specific antibodies, each in the presence of N. gonorrhoeae expressing either Opa₅₀ or Opa₅₂, or CEACAM-specific or control antibodies. In each condition, infection with gonococci expressing the HSPG-specific Opa₅₀ variant increased the proliferation of T cell cultures compared to the uninfected control (FIG. 4). In contrast, gonococcal expression of the CEACAM-specific Opa₅₂ consistently abrogated this effect, generally limiting proliferation to amounts observed in uninfected samples (FIG. 4; IL-2+CD3, CD3+CD28 and IL-2+CD3+CD28). In the case of stimulation by CD3ε-ligation in the absence of IL-2 or anti-CD28 antibodies, Opa₅₂ completely inhibited growth of the lymphocyte culture (FIG. 4, CD3). In each case, CEACAM-reactive or control antibodies had effects similar to that observed for the Opa₅₂- or Opa₅₀-expressing bacteria, respectively, suggesting that the suppression of T cell growth by Opa₅₂-expressing gonococci is due to the bacteria's ability to ligate CEACAM1.

CEACAM1 Ligation does not Increase Lymphocyte Death

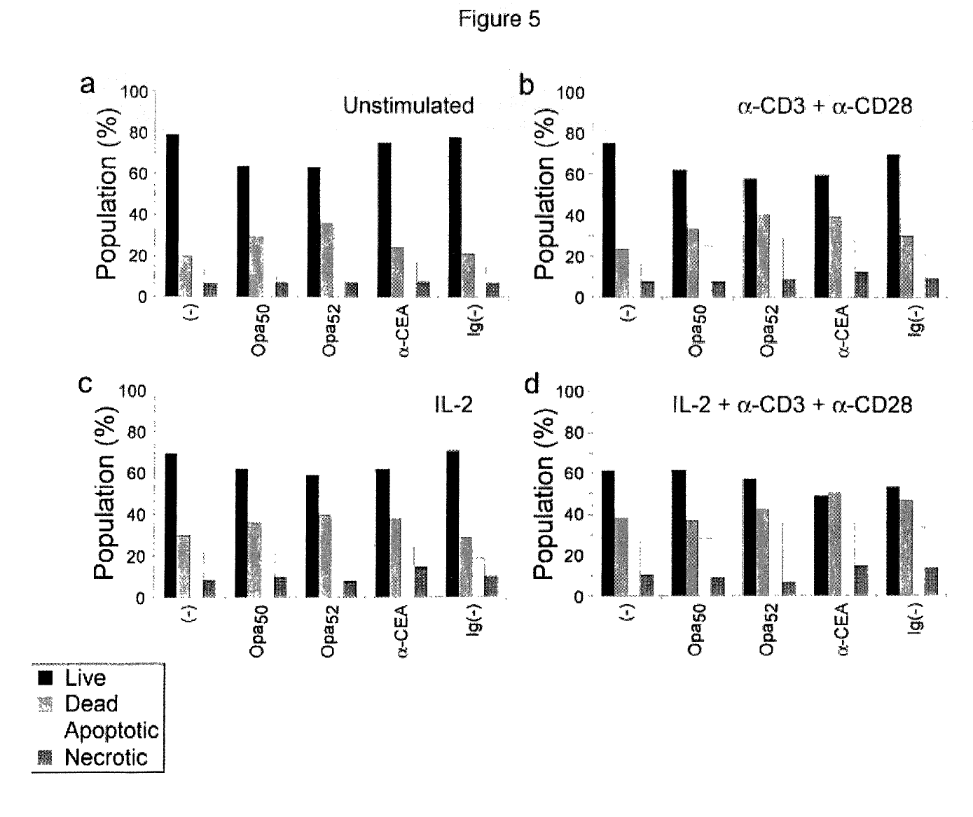

The lower number of activated and proliferating lymphocytes present in samples that contained either Opa₅₂-expressing gonococci or CEACAM-specific antibody could result from either an increase in lymphocyte death, or a decrease in the rate of proliferation among an otherwise viable population. Therefore, the inventors characterized and quantified the effects of gonococcal infection and immunological challenge on lymphocyte viability to determine whether ligation of CEACAM1 increased cell death. In general, different stimuli influenced lymphocyte viability even in the absence of gonococcal infection or immunological challenge (compare (−) samples in FIGS. 5A-D). Specifically, costimulation, through CD3ε and CD28, marginally reduced cell viability, consistent with the fact that profound lymphocyte activation can induce cell death (37). However, after 48 h no strain- or antibody-dependent differences were apparent, regardless of the method of stimulation used (FIG. 5A-D). After 72 h, relative proportions and patterns of cell viability were broadly consistent with the earlier time point, although necrosis was proportionally greater in each activation state (data not shown). Consistent with the observation made after 48 h (FIG. 5), ligation of CEACAM1 by either gonococcal Opa₅₂ or CEACAM-specific antibody failed to induce lymphocyte death relative to the appropriate controls after 72 h (data not shown).

Opa₅₂-bound CEACAM1 Associates with SHP-1 and SHP-2

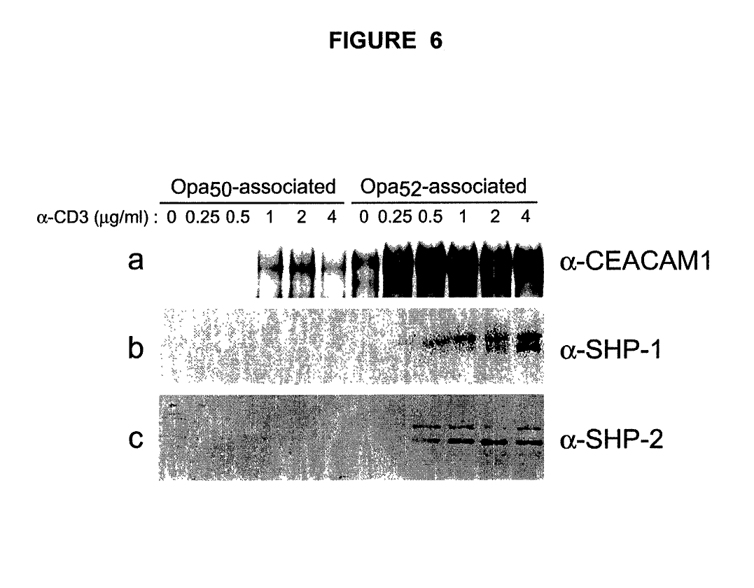

As indicated above, reduced proliferation of CD4⁺ T lymphocytes was not coincident with reduced cell viability. Consequently, the inventors proposed that suppression of lymphocyte activation and proliferation might result from CEACAM1 recruitment of effector molecules, which antagonize otherwise activating stimuli. The association of the tyrosine phosphatases SHP-1 and SHP-2 with ITIM-containing cellular receptors is critical to their function in down-regulating lymphocyte activation (38-40). Therefore, the inventors examined the association of these enzymes with CEACAM1. In order to recover CEACAM1 that was associated with Opa₅₂ rather than total cellular CEACAM1, the inventors developed a ‘bacterial precipitation’. This involved the differential solubilisation of host cell, but not gonococcal membranes, and then centrifugal recovery of intact bacteria with associated host proteins. CEACAM1 was selectively bound by Opa₅₂, with little receptor evident in the pellet containing Opa₅₀-expressing bacteria (FIG. 6A), thus reflecting the established receptor specificities of these Opa variants (14). Consistent with our previous results (FIG. 1), treatment with increasing amounts of cross-linked anti-CD3ε immunoglobulin G (IgG) increased expression of CEACAM1. In this assay, induction was evident with 0.25 μg/ml anti-CD3ε IgG, and maximal expression was induced by 0.50 μg/ml of this antibody (FIG. 6A). No additional increases were noted using higher concentrations of this antibody. SHP-1 and SHP-2 were recovered coincident with CEACAM1, and increasing recovery was evident in the presence of higher concentrations of anti-CD3ε IgG. SHP-1 was coprecipitated following TCR stimulation using 0.5 μg/ml anti-CD3ε IgG, and increased progressively at higher concentrations of this antibody, despite no obvious increase in total CEACAM1 within the bacterial pellet (FIG. 6B). Coprecipitation of SHP-2 was also found under these conditions, however, maximal recovery was achieved following TCR stimulation using 1.0 μg/ml anti-CD3ε IgG (FIG. 6C).

### Discussion

In this study, the inventors have established that infection by gonococci expressing CEACAM-specific Opa proteins suppressed expression of the early activation marker CD69 and the subsequent proliferation of CD4⁺ T cells in response to various activating stimuli. Infection with isogenic strains that do not bind CEACAM instead stimulated the lymphocytes, thereby indicating that the expression of Opa variants that bound to CEACAM1 was required for this effect. Lymphocyte exposure to CEACAM-specific antibodies also suppressed the T cell response, indicating that CEACAM1 ligation alone is sufficient to suppress CD4⁺ T cell activation and proliferation. Furthermore, lymphocyte exposure to a combination of anti-CEACAM1 antibodies and N. gonorrhoeae which was unable to bind CEACAM1, inhibited of lymphocyte proliferation to the same extent as that observed in response to gonococci expressing the CEACAM-specific Opa₅₂ (i.e. in the absence of antibody). Together these findings indicate that Opa-mediated ligation of CEACAM1 is responsible for the gonococci's ability to inhibit CD4⁺ T cell activation and proliferation. These effects were not attributable to strain- or antibody-specific differences in cell viability, or from adhesin or strain-specific differences in bacterial internalisation by the lymphocytes, indicating that the CEACAM1-dependent effects resulted from a specific arrest in cell division rather than from infection-induced cytotoxicity. Such inhibition is consistent with the presence of an ITIM sequence within the cytoplasmic domain of CEACAM1. ITIM phosphorylation allows the recruitment of the SH2-containing phosphatases (38-40), resulting in antagonism of kinase-dependent events, thereby increasing the intensity of the activating stimulus required to induce a lymphocyte response. The inventors observed that CEACAM1 bound by Opa₅₂-expressing gonococci was associated with SHP-1 and SHP-2, suggesting that these tyrosine phosphatases may be involved in the Opa₅₂-dependent suppression of T cell activation and division. Consistent with this, SHP-1 and SHP-2 both contribute to the inhibition of intracellular calcium flux observed in response to ligation of chimeric receptors containing the cytoplasmic tail of CEACAM1 (41).

The inhibitory effect of CEACAM1 ligation, either by CEACAM-specific antibody or gonococci expressing Opa₅₂, was consistently greater following coligation of the ITAM-containing CD3ε chain of the TCR, likely due to increased activity among Src-family kinases, which can phosphorylate CEACAM1 (42). This effect was evident when analyzing CD69 expression and lymphocyte proliferation. When CD3ε-ligation was coincident with the presence of IL-2 and/or CD28-ligating antibodies, the inhibitory effect of CEACAM1 ligation became less dramatic. This is consistent with the threshold activation model, since such costimulation increases the relative magnitude of activating stimulus, thereby overcoming the otherwise inhibitory signal mediated by CEACAM1. While the suppressive effect of Opa₅₂ and CEACAM-specific antibody were still clearly evident in the presence of multiple stimuli, CEACAM1-ligation no longer abrogated activation. In this regard, it should be noted that the inventors used high doses of IL-2 (1000 U/ml) and stimulatory antibodies (1 μg/ml each of anti-CD3ε and anti-CD28) throughout this study. Previous studies show that the coinhibitory effect of other ITIM-containing receptors is more significant if less potent stimulation of the lymphocytes is used (36,43-48), and it is possible that the inhibitory effect of CEACAM1 would be even more pronounced under such conditions. Consequently, our ongoing studies aim to ascertain the effect of Opa₅₂-expressing bacteria and CEACAM1-specific antibody on the response of CD4⁺ T lymphocytes exposed to antigen presented in the context of major histocompatibility complex (MHC) class II.

While the results clearly demonstrated an inhibitory role for CEACAM1, others have reported the opposite effect. Ligation of CEACAM1 has been shown to enhance the proliferation and IFN-γ release by primary lymphocytes (31,32). In contrast, the inventors have observed that the CEACAM-specific Opa₅₂ protein expressed on the surface of N. gonorrhoeae and CEACAM-specific antibody each suppressed T cell activation and proliferation in response to IL-2, CD3ε and/or CD28 receptor-mediated stimulation. Such an inhibitory role is consistent with the ability of CEACAM1 to block the growth of transformed cells (30) and to down-regulate the cytolytic function of intestinal intraepithelial lymphocytes (33). Furthermore, ligation of chimeric receptors that contain the cytoplasmic domain of CEACAM1 inhibits the calcium flux otherwise apparent following B cell receptor ligation (41). Such an effect has been used to help establish the inhibitory role of other ITIM-containing receptors (47,49). The apparent contradictions associated with CEACAM1 are not without precedent in the analysis of lymphocyte function. Depending on the conditions used, the ITIM-containing receptors CD5 (46,47), CD72 (45,50), and PECAM1 (36,49) have each been described as mediating both the activation and inhibition of cellular responses. Consequently, receptor density, degree of cross-linking, nature of the cross-linking ligand, and/or the pre-existing state of cellular activation may each contribute to the apparent function of these coinhibitory receptors.

CD4⁺ lymphocytes are often overlooked as a normal and significant constituent of the sub-mucosa, yet their density is roughly equivalent to that of CD8⁺ lymphocytes in the endocervix (51): CD4⁺ T cells normally constitute ˜2.5% of all cells recovered by endocervical cytobrush, with further recruitment occurring coincident with non-ulcerative sexually transmitted diseases, including gonococcal infection (51). Since gonococci are evident in the sub-epithelial spaces following infection (52), they undoubtedly come into direct contact with these cells. Such interactions would presumably allow Opa binding to CEACAM1, since ˜94% of gonococcal isolates obtained from mucosal infections recognize CEACAM1 (20). The Opa-CEACAM1-induced immunosuppression described herein may thus have several potential benefits to N. gonorrhoeae. CD4⁺ T lymphocytes effectively control the development of a humoral response. Inhibiting the activation and proliferation of CD4⁺ T cells should diminish available T cell help for B cell activation, thus reducing and/or delaying the development of a specific immunity. This may explain why local and systemic antibody responses to gonococcal infection are unexpectedly low and lack signs of developing immune memory (7,8). It is also important to reiterate that CEACAM1 is not restricted to CD4⁺ T cells, but is also expressed by other lymphocytes and professional phagocytes (21,31). Whether Opa-dependent ligation of CEACAM1 also influences the activity of these cells during gonococcal infection remains to be investigated. While the lack of a non-primate animal model precludes simple assessment of the impact of Opa-CEACAM1 interactions in vivo, the ability of an ITIM-containing receptor to suppress an immune response in vivo has been demonstrated: immune complex-induced inflammation is controlled by the relative intensity of activating and inhibitory signals emanating from the ITAM-containing Fcγ versus ITIM-containing FcγRIIB receptors, respectively (55). These findings have lead to the suggestion that targeted induction of ITIM-mediated inhibitory processes should provide a therapeutic strategy by which to impede undesirable inflammatory responses, such as those that occur during autoimmune disease (55). With respect to N. gonorrhoeae binding to CEACAM1, even a short delay in the initiation of an immune response could potentially increase the likelihood that the infecting bacteria successfully colonizes the urethral or cervical mucosa and persist for an extended time. A longer delay may facilitate asymptomatic persistence of N. gonorrhoeae if the intense inflammatory response that typically characterizes gonorrhea is prevented. Infection-induced immunosuppression could also help explain why gonococcal infection increases an individual's risk of acquiring other STD's, including chlamydia (11) and HIV (12), if Opa-dependent interactions also affect the local response to coincident infections. Particularly interesting in this regard are reports that, in HIV-infected individuals, CD4⁺ T cell counts (10) and HIV-1-specific CD8⁺ T cell responses (R. Kaul et al., manuscript in preparation) decline during episodes of gonococcal infection in HIV-1-infected individuals, since it is enticing to speculate that Opa binding to CEACAM1 may influence both of these parameters in vivo. Due to the strict host specificity of N. gonorrhoeae for humans, our ability to dissect the contribution of Opa-CEACAM1 interactions in vivo awaits the generation of transgenic mice that express human CEACAM1 (53) and/or the use of recombinant strains which express Opa variants of defined receptor specificity in the human male urethral challenge model (54).

In conclusion, this study demonstrates the immunosuppressive effect resulting from bacterial engagement of a coinhibitory receptor. The implications of this work are not, however, restricted to gonococcal disease, since Neisseria meningitides (56) and Haemophilus influenzae (57), both of which colonize the upper respiratory tract and can cause invasive disease, also express adhesins that bind CEACAM1. It thus seems likely that such an effect has contributed to the evolutionary success of each of these important human pathogens.

### Example 2

Ligation of CEACAM1 can Arrest the Proliferation of Jurkat CD4⁺ T Lymphocytes

In this Example, the inventors have examined the role of CEACAM1 as a potential ITIM receptor and, specifically, its putative function in the growth of the immortalized Jurkat [JK] CD4⁺ T lymphocyte cell line in vitro. Jurkat cells are routinely used for the in vitro study of T cell receptor-induced signal cascades. Their use has the benefit over using primary cells that it provides large numbers of a homogenous cell population with which to work. However, they do proliferate even in the absence of activating stimuli because they are an immortalized cancer cell line. This allows the influence of various stimuli on the growth of a cancer cell in vitro. While CEACAM1 expression has previously been shown to suppress tumor cell growth (61-65), the proposal that the ligation of CEACAM1 will impede growth of a transformed cell that expresses this receptor is, to our knowledge, a novel one.

JK Cells Express CEACAM1

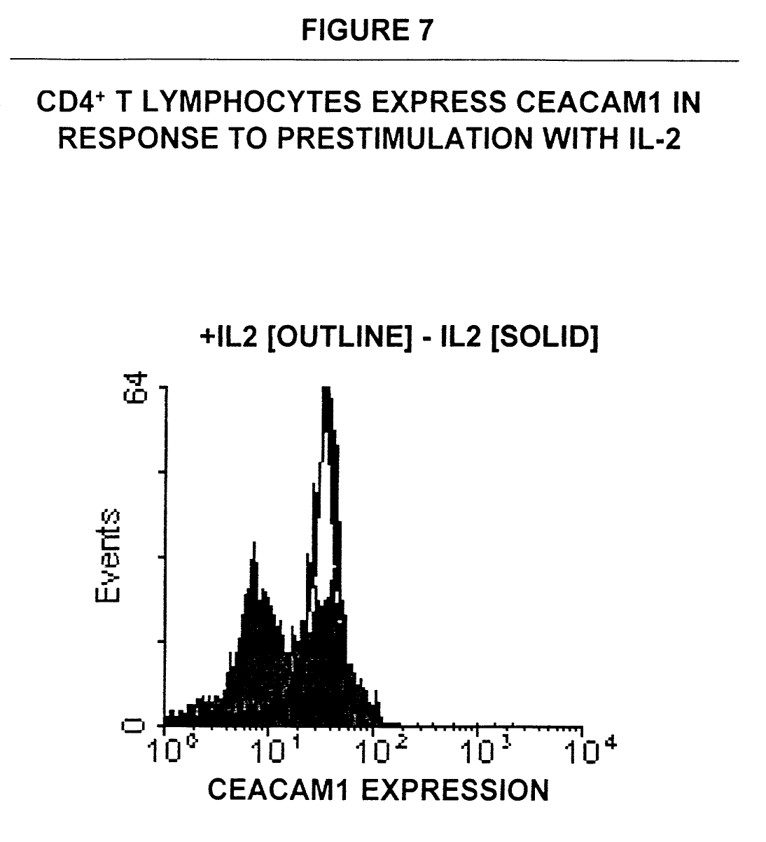

While primary lymphocytes do express CEACAM1, Jurkat cells were previously reported to lack this receptor (31). However, the inventors have found CEACAM1 expression levels were increased by prestimulation with IL-2 at 1000 U/ml 48 h (FIG. 7 and data not shown). In IL-2-stimulated JK cells, degranulation with PMA induced a CEACAM1 phenotype comparable to unstimulated cells (data not shown). This suggests either release or enzymatic degradation of this receptor.

Each GC Test Strain Bound to JK Cells. Prestimulation with IL-2 Enhanced Binding by Strains that Express CEACAM1-binding Opa Proteins

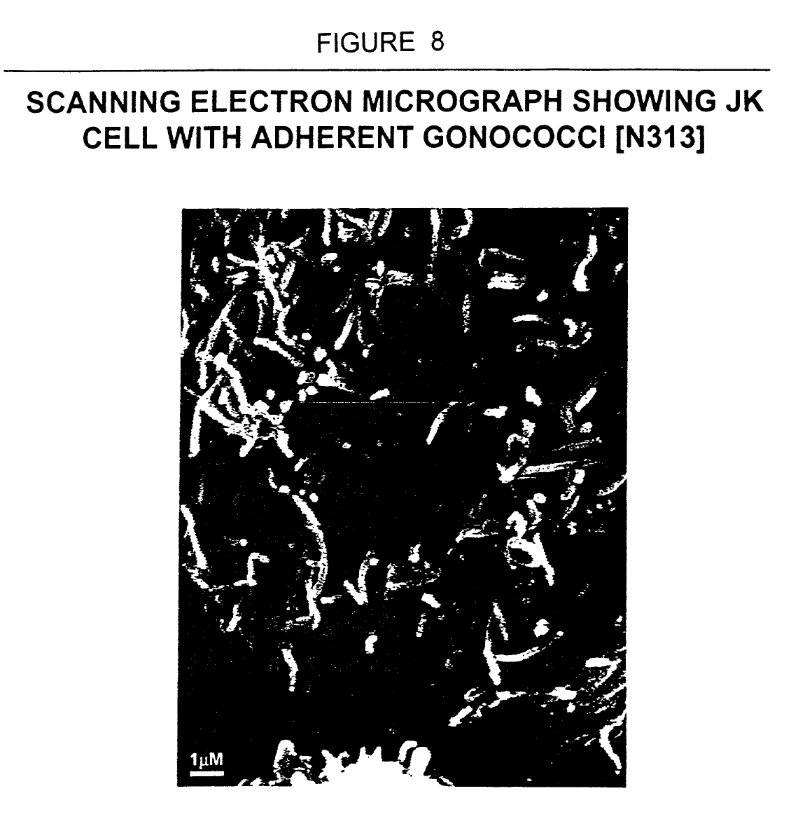

N. gonorrhoeae strains N309 and N313, which express CEACAM-binding Opa₅₂ and Opa₅₇ variants, respectively, formed dense micro-colonies on the surface of Jurkat cells (FIG. 8), possibly suggesting ligand-induced receptor “capping.” This phenomenon may influence intracellular signal transduction by concentrating the inhibitory receptor at foci of bacterial attachment.

Quantification of Adherent Gorococci Recovered from JK cells

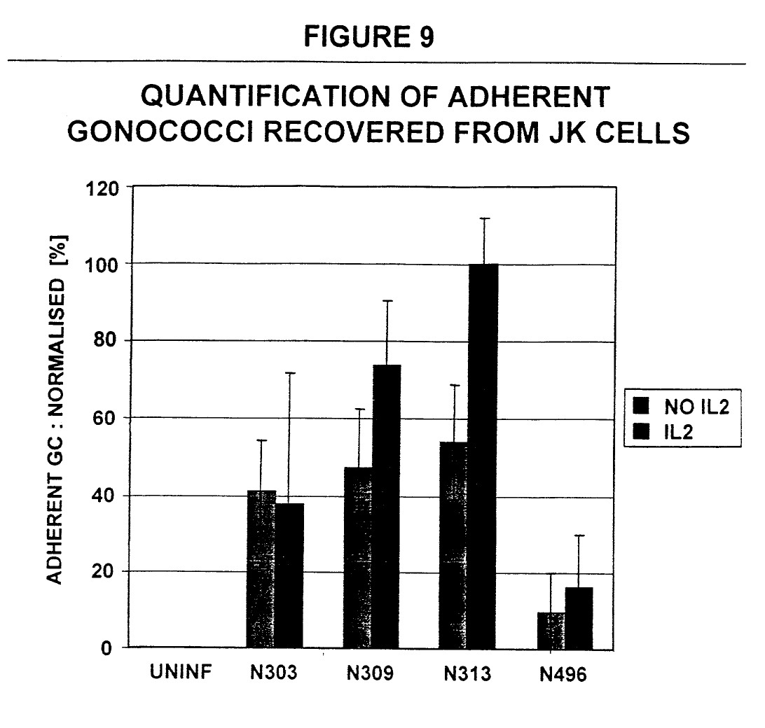

FIG. 9 shows that each GC test strain adhered to JK cells. Binding by strains N309 and N313 which express CEACAM1 ligands Opa₅₂ and Opa₅₇, respectively, increased in response to IL-2 prestimulation, coincident with enhanced CEACAM1 expression. These strains formed dense micro-colonies on the surface of JK cells (see FIG. 8). Note: <1.0% of JK cells had intracellular bacteria as determined by bacterial recovery assay and fluorescence microscopy.

The Proliferation of IL-2 Prestimulated JK Cells was Significantly Reduced by Infection with CEACAM1 Ligand Strains, or Exposure to Anti-CEACAM1 Serum

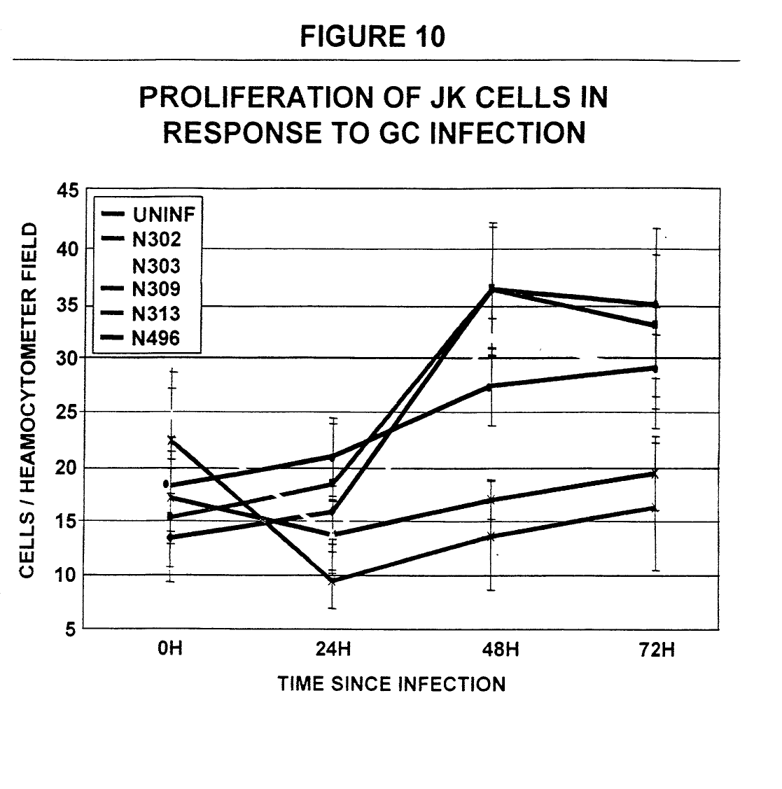

FIG. 10 shows that infection with N309 or N313 (i.e. strains for which CEACAM1 is an adhesion ligand) inhibited the proliferation of JK cells. This effect was dependent on prestimulation with IL-2 which causes increased CEACAM1 expression (see FIG. 7). Gentamicin was added after 5 h to prevent bacterial overgrowth and non-specific toxicity. Bars represent +/− SD (n>10).

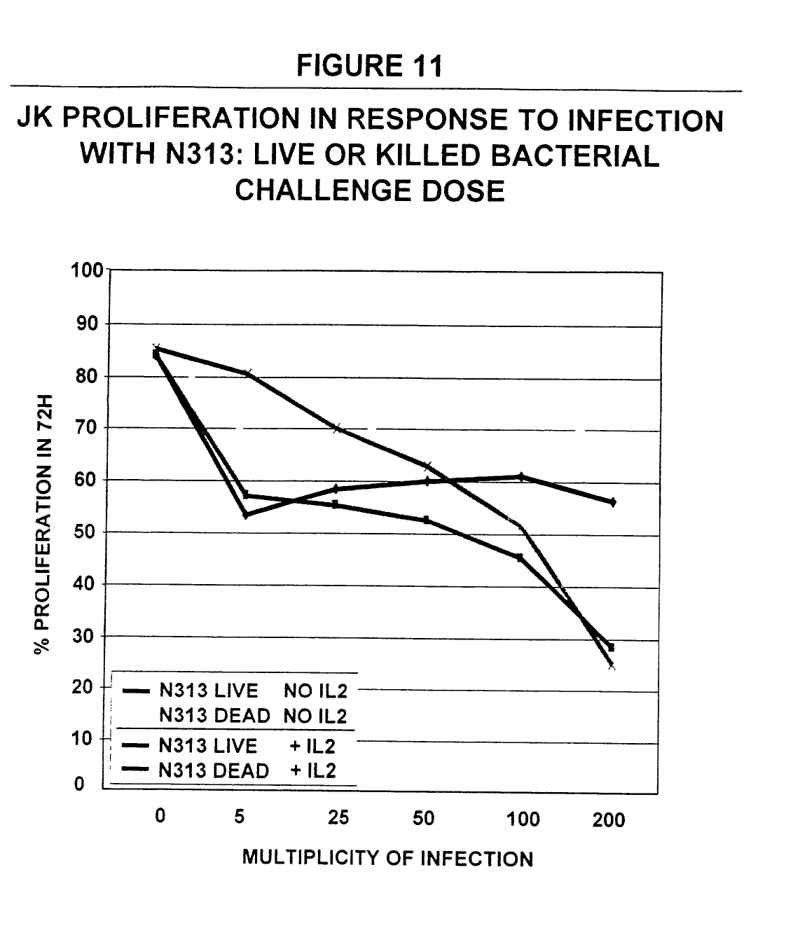

FIG. 11 shows N. gonorrhoeae expressing the CEACAM-specific Opa₅₇ (N313) inhibited the proliferation of IL-2 prestimulated JK cells in a dose-dependent manner. This effect occurred irrespective of initial bacteria viability. These data indicate relative proliferation 72 h post infection.

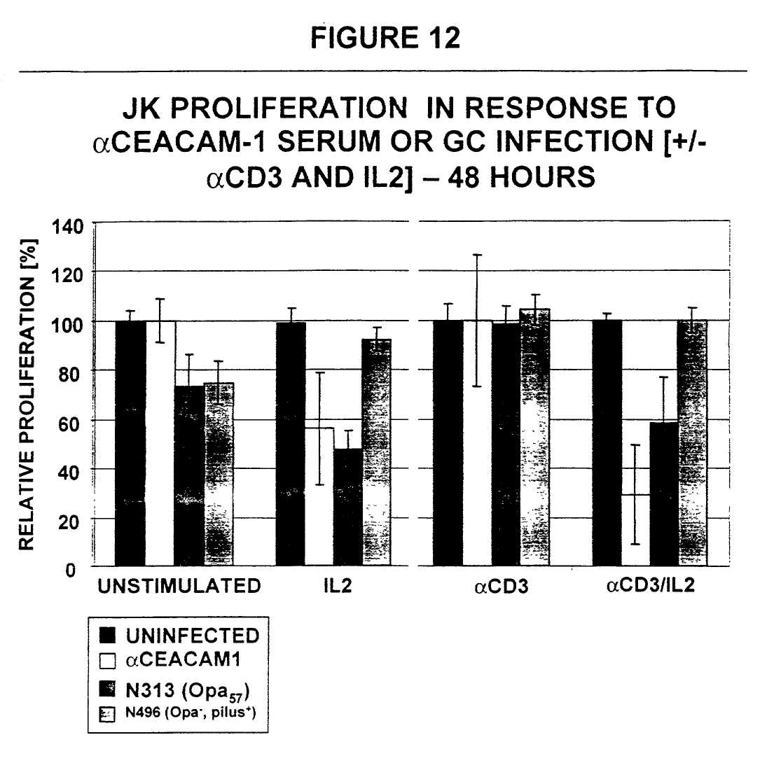

FIG. 12 shows that exposure to anti-CEACAM1 serum or N. gonorrhoeae expressing CEACAM1-binding Opa₅₇ (N313) inhibited the proliferation of IL-2 stimulated JK cells as compared to uninfected cells or cells that were infected with a gonococcal strain that does not express an Opa protein but does express the pilus adhesion (N496). Inhibition was demonstrable and consistent irrespective of T cell receptor co-stimulation using anti-CD3 IgG which was cross-linked using F(ab)₂ anti-mouse IgG serum. Note: These data represent an infectious MOI=200 and an antibody concentration of 50 μg/ml. However, inhibitory effects were apparent at MOI=25 and an antibody concentration of 5 μg/ml respectively.

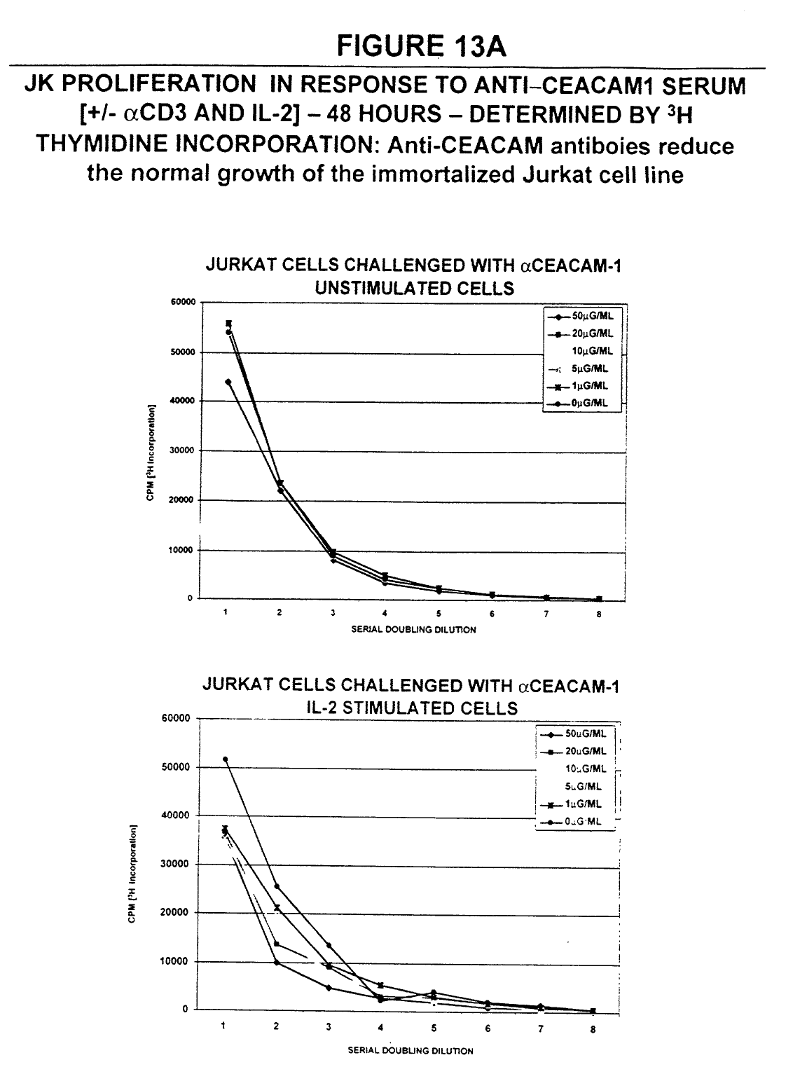

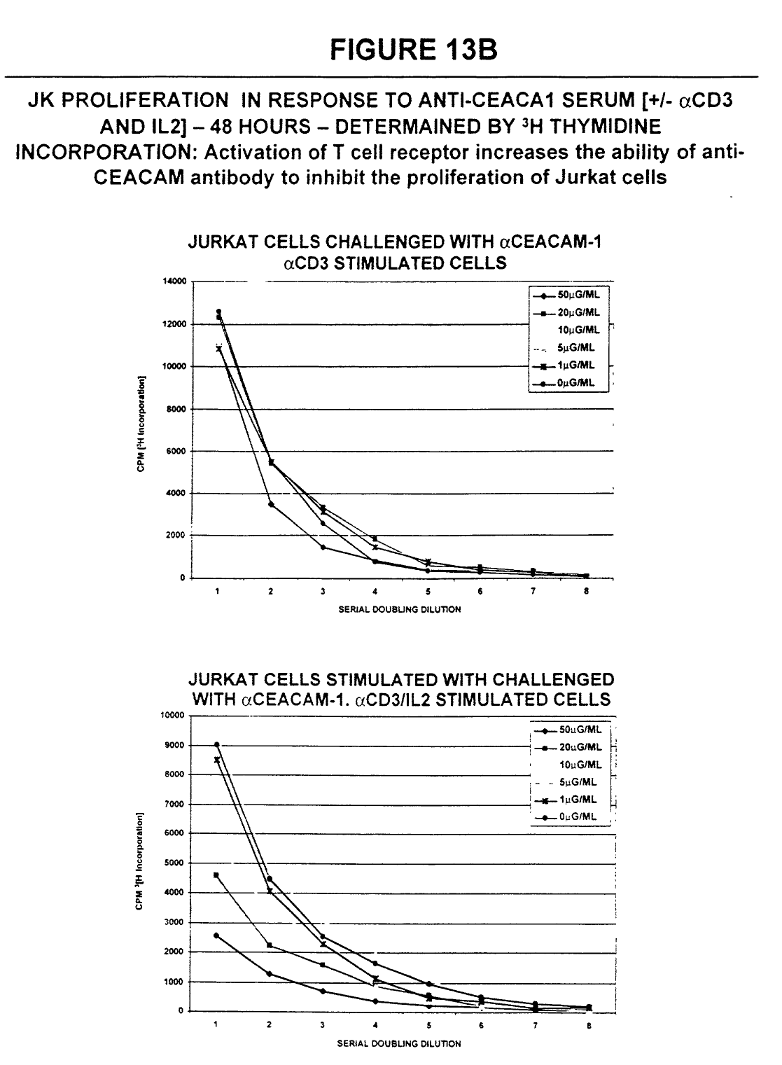

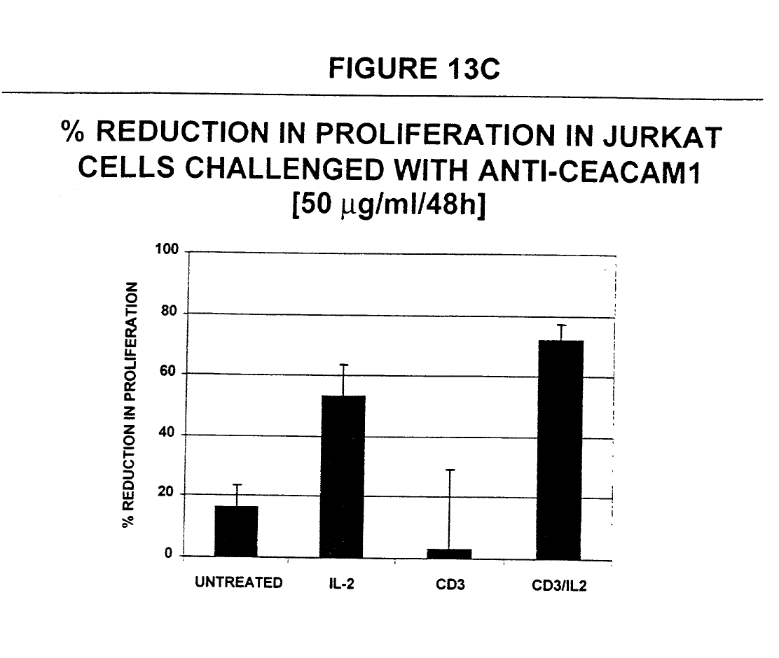

FIG. 13A shows that anti-CEACAM antibody reduces the normal growth of the immortalized Jurkat cell line. In FIGS. 13B and C, lymphocyte proliferation was assessed by thymidine incorporation 48 h after immunological challenge. Each sample was pulsed with (1 μCu ³H) thymidine for 5 h, and serial doubling dilutions were prepared. Lymphocytes in each sample were harvested onto 96 well glass-filter plates and radio-nucleotide incorporation was assessed by liquid scintillation counting. Together, these data indicate that ligation of CEACAM1 resulted in an inhibition of proliferation of Jurkat (JK) cells in vitro. In the absence of prior stimulation this effect required antibody concentrations of 50 μg/ml. However, JK cells prestimulated with IL-2 (and therefore expressing more CEACAM1) were inhibited in a dose dependent manner (minimum inhibitory antibody concentrations of 1 μg/ml). This effect was reduced in JK cells stimulated with anti-CD3 (crossed linked with goat anti-mouse F(ab)₂). However, costimulation with anti-CD3 and IL-2 significantly increased the apparent inhibitory effect of CEACAM1 ligation, and further, increased the apparent titration effect (see previous figures). This is consistent with anti-CD3 inducing a TCR-associated ITAM response, which involves the activation of Src family kinase that phosphorylates tyrosine residues within the CEACAM1 ITIM. This would allow recruitment of phosphatases which reduce the proliferation signal. These results also suggest that IL-2 treatment of the cells allows the inhibitory function of CEACAM1 to occur. The inventors have seen that IL-2 does increase CEACAM1 expression by Jurkat cells ˜2-fold, however whether this is enough to explain the effect of IL-2 on CEACAM1-mediated inhibition of cell growth is still uncertain. Together, these data suggest that CEACAM1 can inhibit the normal growth of the immortalized Jurkat cell line and can inhibit the activation-induced proliferation of Jurkat cells.

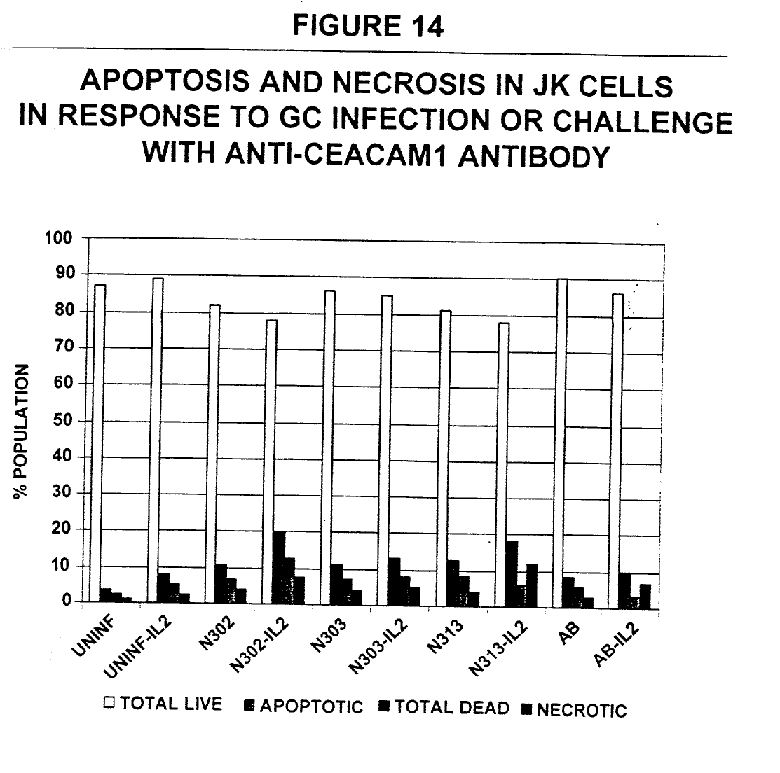

FIG. 14 shows that neither infection nor CEACAM1 antibody (AB) challenge significantly increased total cell death, apoptosis, or necrosis in the Jurkat challenge model. This indicates that proliferative inhibition results from cellular “arrest” rather than “toxicity”.

In each case inhibition was dose dependent, occurred irrespective of bacterial viability and did not induce selective toxicity. These data indicate that CEACAM1 acts as an inhibitory receptor in JK cells and, furthermore, that gonococcal infection may have a growth-inhibitory effect.

### Example 3

A Phage Display-based Approach to Assess CEACAM Receptor Binding by Neisserial Opa Protein-derived Peptides

In this Example, the inventors have demonstrated that linear peptide fragments from neisserial Opa proteins are sufficient to mediate CEACAM-specific binding. The colony opacity-associated (Opa) proteins are integral outer membrane proteins expressed by pathogenic and commensal Neisseria sp. (14,76). Most Opa variants tested mediate attachment to human cells via either heparan sulfate proteoglycan-containing cell surface receptors and/or the carcinoembryonic antigen-related cellular adhesion molecules (CEACAMs; see ref. 14 and Table 1). Recombinant bacteria that express distinct Opa variants can bind soluble or cell surface-expressed HSPG or CEACAM receptors and are efficiently engulfed by cells that express one or both of these receptor types (14). Example 1 shows that expression of CEACAM1-specific Opa variants allows the bacteria to suppress CD4⁺ T cell function.

In this Example, the inventors used a phage display-based approach to ascertain whether isolated Opa protein fragments can bind to CEACAM-family receptors. Membrane-spanning regions of the protein are highly conserved, while the external loops may display regions of high variability: Loop 1 displays a semi-variable region (SV), loops 2 (HV1) and 3 (HV2) contain hyper-variable regions, whereas loop 4 (C-L) is highly conserved (66,58,74). The inventors have generated oligonucleotide primers complementary to the conserved nucleotide sequences that encode transmembrane or membrane-proximal sequences of Opa. These were used to amplify gene fragments encoding single surface-exposed loops from the 11 well-characterized opa alleles encoded by Neisseria gonorrhoeae strain MS11. Four pools of PCR products were generated by combining gene fragments that encode corresponding loops each individual allele. Each pool was then cloned into a surface-exposed portion of the pVIII coat protein of M13 filamentous bacteriophage. By sequential panning over stably transfected cell lines expressing known cellular receptors, the inventors were able to recover recombinant phage that bound specifically to HeLa-CEACAM, but not to the parental HeLa-Neo cell line that lacks CEACAM but does express HSPG receptors. CEACAM-specific phage were recovered from the HV1 and HV2 phage pools, while neither wild type phage nor those displaying other Opa loops were recovered from the CEACAM-expressing cells. These data indicate that subfragments of one or more Opa proteins are sufficient to mediate CEACAM binding, and supports the feasibility of using Opa fragments as a means by which to specifically target this receptor family. This is, to the inventors' knowledge, the first demonstration that subfragments of Opa are able to mediate binding to CEACAM family receptors.

### Materials and Methods

Bacterial Strains and Cell Lines

E. coli DH5 strains with Hermes 10-based vectors containing individual opa alleles derived from N. gonorrhoeae MS11 (denoted as opa50, opa51, . . . opa60) were previously described by Kupsch et al. (1993) (58). These strains were cultured on LB agar plates (LB medium, 15 g/L bacto-agar) supplemented with 25 μg/ml kanamycin. E.coli TG-1 (75) were routinely grown on 2×YT medium (16 g bacto-tryptone, 10 g bacto-yeast extract, 5 g NaCl per litre, adjusted to pH 7.0 with 5 N NaOH). Where appropriate, the medium was supplemented with 50 μg/ml ampicillin to select for transformant colonies.

HeLa cells used in phage panning have been described previously (70), and were propagated in RPMI 1640 medium (Invitrogen Life Technologies, Canada) supplemented with 10% heat-inactivated Fetal Bovine Serum (FBS; Invitrogen), and maintained at 37° C. with a 5% CO2 humidified atmosphere.

Transformation Conditions

Transformation of E. coli DH5 was performed using calcium-induced bacterial competence, as described by Sambrook et al. (1989) (75). To transfer plasmid into TG-1 cells, 5 μl of mini prep recombinant plasmids isolated from the DH5 transformants was used to transform electroporation competent (75) TG-1 cells that had been pre-chilled in 0.2 cm electroporation cuvettes (Bio-Rad) using the Bio-Rad Gene pulser set at a voltage of 2.5 kV, capacitance of 25 μF, and a resistance of 200 Ω.

Plasmids

Plasmid DNA used as template in PCR reactions was obtained from E. coli DH5 cells by boiling prep lysis. Single colonies were selected with a sterile wooden toothpick and resuspended in 50 μl of autoclaved ddH₂0. The sample was placed in boiling water for 5 minutes, and then centrifuged for 3 minutes at 13,000 rpm. 5 μl of supernatant was used as template in subsequent PCR reactions. pG8ASET phagemid DNA was isolated from TG-1 cells by the CONCERT standard midi preparation kit (Invitrogen). All other plasmid DNA used for transformation and/or recombinant cloning was isolated from E. coli using a plasmid mini preparation method (75). Single colonies were inoculated into 2×YT media and grown overnight at 37° C. 1.5 ml of culture was pelleted by centrifugation 13,000 rpm for 2 minutes. The pellet was resuspended in 350 μl STET and 25 μl of lysozyme solution (10 mg/ml in 10 mM Tris-Cl pH 8.0) was added to the sample. The sample was placed in a boiling water bath for 40 seconds and subsequently centrifuged at 12,000 rpm for 10 minutes. Nucleic acid was then precipitated by the addition of 150 μg 7.5 M ammonium acetate and 600 μl of isopropanol and then incubating in a dry ice/ethanol bath for 30 minutes before centrifugation at 12,000 rpm. The supernatant was discarded, 1 ml of 70% ethanol was added and centrifuged at 12,000 rpm for 2 minutes at 4° C. The pellet was then re-dissolved in 100 μl ddH₂0.

The pG8ASET phagemid vector previously described by Frykberg and Jacobsson (73) was used to construct the opa gene fragment libraries. The double stranded phagemid is approximately 3.4 kB, with a protein A leader sequence, multiple cloning site, and E-tag peptide fused to the M13 phage coat protein pVIII. The E-tag/pVIII fusion protein was inserted with a frameshift such that it will be expressed only when the reading frame has been corrected by insertion of a foreign sequence. The pG8ASET vector also contains a suppressible stop (TAG) codon between the E-tag and the pVIII coat protein, which allows for screening of transformants in E.coli DH5 cells for E-tag expression without expression of the entire pVIII coat protein (73). TG-1 cells are transformed for phagemid production, as they possess the supE/F mutation that suppresses the TAG stop codon.

Generation of Recombinant Phage Library

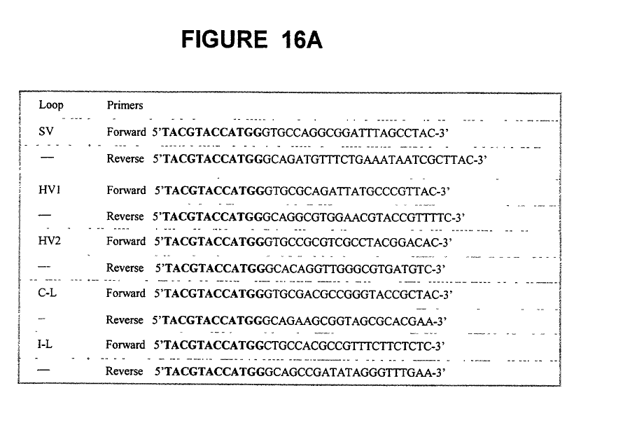

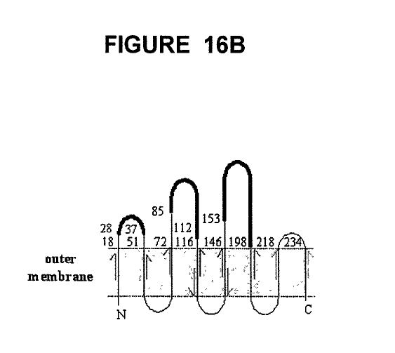

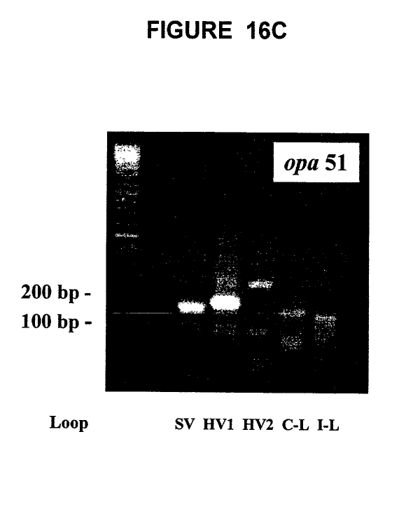

Gene fragments encoding Opa sequences predicted to include single surface-exposed loops were amplified by PCR from Hermes 10-based vectors containing individual opa alleles from N. gonorrhoeae strain MS11 (58). Five reactions were performed for each allele using different sets of primers, each primer being designed to anneal with conserved sequences encoding transmembrane or membrane-proximal sequences so that corresponding pairs will specifically amplify single loops (FIG. 16). A cysteine codon (5′-TGC-3′) was added to each oligonucleotide primer sequence to allow constriction of the inserted Opa fragment by facilitating the formation of a novel disulfide bond between cysteine residues that flank it. An Ncol restriction endonuclease cleavage site (5′-CCATGG-3′) was also added distal to the opa coding sequence in each primer to allow the subcloning of fragments into the pG8ASET phagemid vector. The reactions contained 5 μl of template, 0.2 mM dNTPs, 5 μl DMSO, 10 μl 1×ThermPol-buffer (New England BioLabs), 1 μl VENT polymerase (2000 U/ml) (New England Biolabs), and 10 ml 100 μM MgSO₄ in a total reaction volume of 50 μl. Reaction conditions for PCR were 90° C. for 10 min, followed by 30 cycles of 95° C. for 30 s, 52° C. for 60 s, and 72° C. for 30 s. The reaction was then extended for 10 minutes at 72° C. and stored at 4° C. Resulting products were analyzed by gel electrophoresis using a 2.0% agarose gel stained using ethidium bromide.

Individual loops were cloned into pG8ASET by digestion of the PCR products and isolated phagemid vector with Ncol, using the manufacturer's protocol (New England BioLabs). DNA was recovered by ethanol precipitation and then resuspended in 20 μl ddH₂O. The pG8ASET vector was treated with calf-intestinal phosphatase (CIP; New England Biolabs) and incubated for 60 minutes at 37° C. The vector was then electrophoresed and gel purified using the QIAEX II gel purification kit (Qiagen, Mississauga, Ontario). Digested PCR product from corresponding loops were combined to generate four pools that each contain species derived from multiple variants encoding each Opa loop. Such pools were then ligated into the pG8ASET phagemid vector in a 5:1 insert:vector ratio using 4.0 Weiss units of T4 ligase in 1X ligase buffer (New England BioLabs) at 14° C. overnight.

Following transformation, TG-1 cells were selected in 2×YT-G with 50 μg/ml ampicillin broth overnight (37° C.). After estimation of cell density by OD at 540 nm, the cultures were infected with the M13K07 helper phage at multiplicities of infection (MOI) of 50, 100, 500 and 5000. 100 ml of infected culture was mixed with 3 ml of top agar consisting of 0.5% bacto-agar in 2×YT medium, and the mixture then used to cover 2×YT-Amp agar plates. Following 10 hours incubation at 37° C., recombinant phage particles were isolated by removing the top agar into 3 ml of 2×YT followed by agitation at 4° C. overnight. The mixture was centrifuged at 12,000 rpm for 30 minutes at 4° C. The resulting supernatant was then filter-sterilized by passing it through a 0.2 micron filter disc.

Screening of Transformants

Blotting—Transformed bacteria recovered following selection on 2×YT medium with 50 μg/ml ampicillin were screened based on a method by Sambrook et al. (1989) for screening bacterial colonies. Plates were overlayed with nitrocellulose filters, and the colonies then lifted by removing the filters. The filters were then exposed to chloroform vapor for 15 minutes and then incubated with lysis buffer (100 mM Tris-Cl (pH 7.8), 150 mM NaCl, 5 mM MgCl₂, 1.5% BSA, 1 μg/ml DNase I, 40 μg/ml lysozyme) overnight at RT with shaking. Resulting colony blots were blocked using TNT (10 mM Tris-HCl (pH 8.0), 150 mM NaC, 0.05% Tween 20) buffer with 5% non-fat milk, and then probed with an E-tag specific monoclonal antibody (mAb; Pharmacia Biotech, Baie d′Urfe', Quebec). Membranes were washed with TNT, exposed to 20° goat anti-mouse alkaline phosphatase-conjugated antibody and then developed by pre-incubation with 100 mM Tris-HCl (pH 9.5) followed by the addition of BCIP/NBT developer (1 μl Tris-HCl (pH 9.6), 70 μl 1 M MgCl₂, 100 μg/ul 5 μg/ul BCIP, 200 μl 5 μg/gl NBT).

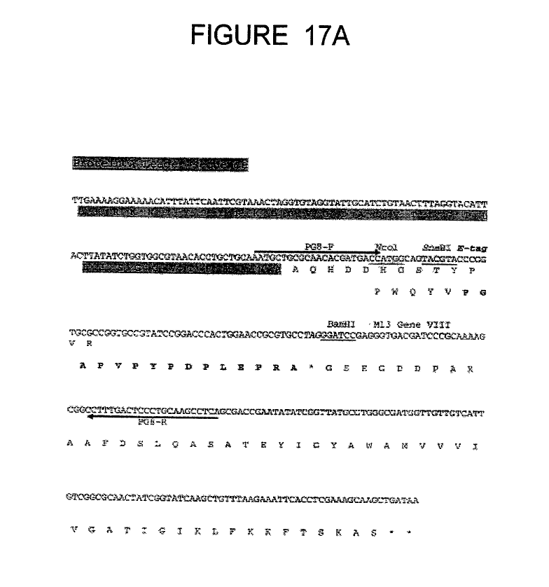

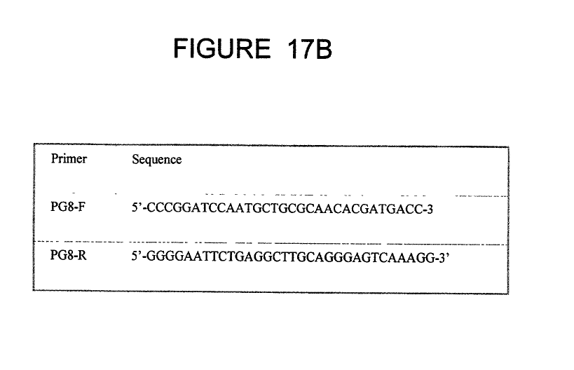

In some cases, colonies were also screened for insert by PCR using plasmid isolated from single colonies by boiling prep. The primers PG8F and PG8R, which flank the insert site and E-tag-encoding sequence (FIG. 17), were used to examine if selected colonies contained the inserted loops. The reaction conditions for PCR were 90° C. for 10 minutes, followed by 30 cycles of 95° C. for 60 sec, 57° C. for 90 sec and 72° C for 90 secs. The reaction was then extended for 10 minutes at 72° C. and stored at 4° C. The resulting products were identified based on size, as seen by 2.0% agarose gel electrophoresis.

Recovery of CEACAM-specific Recombinant Phage

Detection of CEACAM-binding function conferred by expression of the recombinant Opa fragments was performed by sequential panning of recombinant phage particles over the Hela cell lines that either do not (Hela-Neo) or do (Hela-CEACAM) express CEACAM receptors. Recombinant phage particles were added to 3 ml of RPMI 1640 without FBS, which was then used to inoculate 9.6 cm² wells containing ˜50% confluent Hela-Neo cells. The infected cells were agitated by shaking for 1 hour at RT, and culture supernatants which contain unbound phage, were collected. Supernatants were then transferred into wells containing HeLa-CEACAM, and these were incubated together for 1 hour RT with shaking. The supernatant was discarded, and the cells then washed 20 times using phosphate-buffered saline containing 0.05% Tween 20 (PBST). Bound phage were eluted using 200 μl of 0.1 M glycine, pH 2.3 and then immediately neutralized with 2 M Tris-HCl, pH 8.6. Eluted phage were titred and amplified by infecting TG-1 cells, plating on 2×YT containing 50 μg/ml ampicillin, and then colony blotted to detect E-tag expression.

### Results and Discussion

The nucleotide sequence and receptor specificity of each Opa variant encoded by N. gonorrhoeae strain MS11 has been described (14). Despite this fact, no conserved, surface-exposed sequences that correlate with receptor specificity are apparent. The binding function of chimeric Opa proteins and Opa deletion mutants generated from several opa variants has revealed that the HV-1 of Opa₅₀ was required for HSPG receptor binding. While the other variable domains are not required for HSPG binding, each loop does contribute to HSPG binding (69). CEACAM receptor binding was generally less clear, as particular combinations of HV-1 and HV-2 appear to be required for normal CEACAM-binding activity (67). Because of the approach used in these studies, it remains unclear whether these regions bind directly to their respective receptor or, instead, function to maintain a protein conformation required for contact between those sequences that do bind to the receptor.

In order to assess whether isolated fragments of Opa that could mediate CEACAM receptor binding could be recovered, a phage display-based approach was used. The pG8ASET plasmid contains a constitutive protein A leader sequence fused to a multiple cloning site, the E-tag epitope, and a recombinant M13 phage pVIII coat protein (FIG. 17). Peptide fragments inserted into the multiple cloning site are expressed on the surface of recombinant phage particles which retain the peptide coding sequence within their packaged genome. This phagemid vector system and its derivatives have previously been used to screen various random peptide libraries for specific protein-protein interactions (71,72,73,77), including the successful identification of ligand-binding domains from prokaryotic adhesins (71).

The inventors used sequence alignment of the N. gonorrhoeae MS11 Opa variants (58) and the predicted 2-dimensional structure of Opa proteins (74) to design oligonucleotide primers that anneal with the conserved sequences which encode transmembrane and membrane-proximal sequences of the various Opa proteins (see FIGS. 16A-B). PCR amplification of individual Opa loops was carried out using cloned recombinant opa alleles as template, thereby allowing the inventors to confirm amplification of each opa fragment by electrophoretic mobility of reaction products (e.g. FIG. 16C). Analogous loops were combined to generate five pools: SV1, HV1, HV2, C-L or I-L, as appropriate (FIG. 16B), and individual pools were then ligated into pG8ASET. The ligation mixtures were transformed into E. Coli DH5 to generate and allow amplification of the plasmid libraries and screening of transformants for E-tag epitope expression, which denotes insertion of cloned fragments into the gene VIII Ncol cloning site (see FIG. 17). Clones deemed positive by E-tag colony blofting were further screened by PCR with PG-8 forward and reverse primers to confirm insertion of appropriate sized DNA fragments. Individual clones were not isolated, but rather each population was screened to ensure that positive clones were present within the population. These were then used to construct five recombinant (i.e. one four each loop) phage libraries by transferring plasmid to TG-1 cells and then infecting with helper phage.

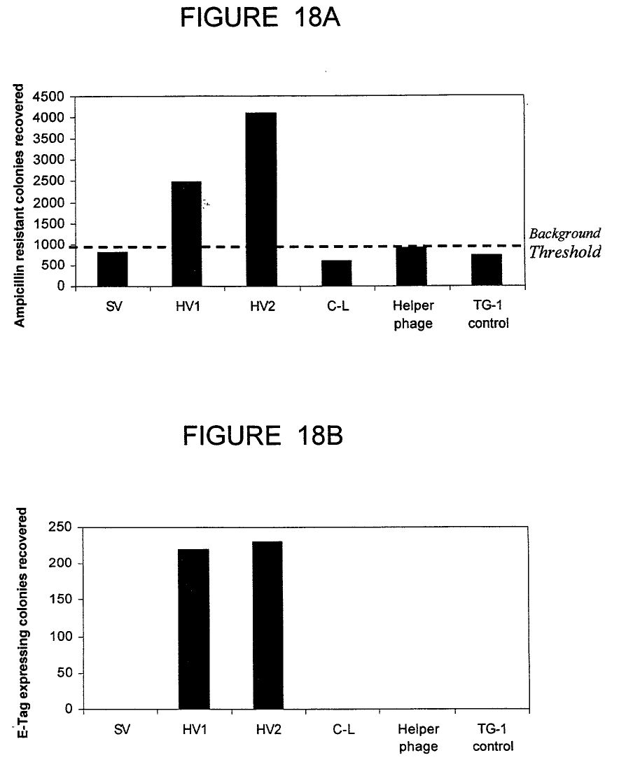

Detection of phage that specifically bind to CEACAM was done by sequential panning of phage particles over two different Hela cell lines. Isolated recombinant phage were initially panned over Hela-Neo cells to remove phage that may bind to cell surface components other than CEACAM, including both specific binding to HSPG-containing receptors and non-specific binding to other cellular receptors and/or extracellular matrix proteins. Phage that remained in the culture supernatant were then panned over the stably transfected HeLa-CEACAM cells. Bound phage particles were eluted and then used to infect TG-1 cells. Recombinant bacteria thus generated were detected by selection for ampicillin resistance, which is conferred by the pG8ASET plasmid that is preferentially packaged in recombinant phage. Recovered colonies were secondarily screened for E-tag expression to confirm the presence of inserted sequence within the phagemid DNA. Based upon both screening techniques, increased binding to HeLa-CEACAM was conferred by expression of sequences contained within the HV1 and HV2 libraries, while binding of SV and C-L libraries was indistinguishable from the control plates that contained panned M13K07 helper phage or uninfected TG-1 cells (FIG. 18). These results indicate that loop 2, which contains HV1, and loop 3, which contains HV2, of one or more Opa variants can mediate specific binding to CEACAM receptors in the absence of other Opa-derived sequences. It is possible that that loops 1 and/or 4 may also contain CEACAM binding activity, however this was not detected by this approach

While the present invention has been described with reference to what are presently considered to be the preferred examples, it is to be understood that the invention is not limited to the disclosed examples. To the contrary, the invention is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

TABLE 1

| | Type | Opa | Opa | Cellular | |
|---|---|---|---|---|---|
| Species | strain | allele^{a} | protein^{a} | receptor | Refs. |
| Neisseria | MS11 | C30/C50^{b} | 30 (50)^{b} | HSPG | van Putten, J. P. and Paul, S. M. |
| gonorrhoeae | | | | | (1995) EMBO J. 14, 2144-2154; |
| | | | | | Chen, T. et al (1995) J. Exp. Med |
| | | | | | 182, 511-517 |
| | MS11 | B51 | 51 | CEACAM5 | Bos, M. P. et al. (1997) Infect. |
| | MS11 | G52 | 52 | CEACAM1,3,5,6 | Immun. 65, 2353-2361; Chen, T. et |
| | MS11 | A53 | 53 | CEACAM1 | al. (1997) J. Exp. Med 185, 1557-1564; |
| | MS11 | I54 | 54 | CEACAM1,5 | Gray-Owen, S. D. et al. |
| | MS11 | E55 | 55 | CEACAM5 | (1997) Mol. Microbial. 26, 971-980 |
| | MS11 | F56 | 56 | CEACAM5 | |
| | MS11 | K57 | 57 | CEACAM1,3,5,6 | |
| | MS11 | J58 | 58 | CEACAM1,3,5,6 | |
| | MS11 | D59 | 59 | CEACAM1,5 | |
| | MS11 | H60 | 60 | CEACAM1,3,5,6 | |
| Neisseria | 00170 | B92 | 92 | CEACAM1,5 | Muenzner, P. et al. (2000) Infect. |
| meningitidis | F6124 | B94 | 94 | CEACAM1,5 | Immun. 68:3601-3607. |
| | F6124 | D100 | 100 | CEACAM1 | |
| | 00170 | J101 | 101 | Unknown^{d} | |
| | 00170 | A132 | 132 | CEACAM1,3,5,6 | |
| | C751 | D2000 | 2000 | CEACAM1^{e} | Virji, M. et al. (1996) Mol. |
| | C751 | A2100 | 2100 | CEACAM1^{e} | Microbial. 22, 941-950 |
| | C751 | B2200 | 2200 | CEACAM1^{e} | |
| | H44/76 | Unknown^{c} | 28^{c} | CEACAM1^{e} | de Vries, F. P. et al (1998) Mol. |
| | | | | | Microbial. 27, 1203-1212 |

^{a}Nomenclature used is as described in Malorny, B. et al. (1998) J. Bacteriol. 180, 1323-1330 and was provided by M. Achtman of the Max Planck Institute for Molecular Genetics, Berlin, Germany. A list of nomenclature for all currently described opa alleles can be found at http://novell-ti.rz-berlin.mpg.de.
^{b}opaC30/Opa₃₀ and opaC50/Opa₅₀ refer to chromosomal and recombinant forms of the same allele, respectively.
^{c}Allele not yet determined because opa sequence currently unavailable.
^{d}Opa₁₀₁ binds neither heparan sulphate proteoglycan (HSPG) nor CD66 receptors, as determined by the interaction of recombinant Escherichia coli strains with stably transfected HeLa cell lines.
^{e}Specificity, for other CEACAM receptors not yet determined.
Table adapted from Dehio et al. (1998) Trends Microbiol. 6:489-495.

Full Citations for References Referred to in the Specification

1. Handsfield, H. H. Neisseria gonorrhoeae in Principles and Practice of Infectious Diseases 1613-1631. Churchill Livingstone, N.Y. (1990).

2. Al-Suleiman, S. A., Grimes, E. M., & Jonas, H. S. Disseminated gonococcal infections. Obstet. Gynecol. 61, 48-51 (1983).

3. Nassif, X., Pujol, C., Morand, P., & Eugene, E. Interactions of pathogenic Neisseria with host cells. Is it possible to assemble the puzzle? Mol. Microbiol. 32,1124-1132 (1999).

4. Plummer, F. A. et al. Antibodies to opacity proteins (Opa) correlate with a reduced risk of gonococcal salpingitis. J. Clin. Investigation. 93, 1748-1755 (1994).

5. Fox, K. K. et al. Longitudinal evaluation of serovar-specific immunity to Neisseria gonorrhoeae. Am. J. Epidemiol. 149, 353-358 (1999).

6. Hobbs, M. M. et al. Molecular typing of Neisseria gonorrhoeae causing repeated infections: evolution of porin during passage within a community. J. Infect. Dis. 179, 371-381 (1999).

7. Hedges, S. R., Sibley, D. A., Mayo, M. S., Hook, E. W., & Russell, M. W. Cytokine and antibody responses in women infected with Neisseria gonorrhoeae: effects of concomitant infections. J. Infect. Dis. 178, 742-751 (1998).

8. Hedges, S. R., Mayo, M. S., Mestecky, J., Hook, E. W., & Russell, M. W. Limited local and systemic antibody responses to Neisseria gonorrhoeae during uncomplicated genital infections. Infect. Immun. 67, 3937-3946 (1999).

9. Kozlowski, P. A., Cu-Uvin, S., Neutra, M. R., & Flanigan, T. P. Comparison of the oral, rectal, and vaginal immunization routes for induction of antibodies in rectal and genital tract secretions of women. Infect. Immun. 65,1387-1394 (1997).

10. Anzala, A. O. et al. Acute Sexually Transmitted Infections Increase Human Immunodeficiency Virus Type 1 Plasma Viremia, Increase Plasma Type 2 Cytokines, and Decrease CD4 Cell Counts. J. Infect. Dis. 182, 459-466 (2000).

11. Hillis, S. D., Nakashima, A., Marchbanks, P. A., Addiss, D. G., & Davis, J. P. Risk factors for recurrent Chlamydia trachomatis infections in women. Am. J. Obstet. Gynecol. 170, 801-806 (1994).

12. Cohen, M. S. Sexually transmitted diseases enhance HIV transmission: no longer a hypothesis. Lancet 351, 5-7 (1998).

13. Fleming, D. T. & Wasserheit, J. N. From epidemiological synergy to public health policy and practice: the contribution of other sexually transmitted diseases to sexual transmission of HIV infection. Sex. Transm. Infect. 75, 3-17 (1999).

14. Dehio, C., Gray-Owen, S. D., & Meyer T. F. The role of neisserial Opa proteins in interactions with host cells. Trends Microbiol. 6, 489495 (1998).

15. Swanson, J., Barrera, O., Sola, J., & Boslego, J. Expression of outer membrane protein 11 by gonococci in experimental gonorrhea. J. Exp. Med. 168, 2121-2130 (1988).

16. Jerse, A. E. et al. Multiple gonococcal opacity proteins are expressed during experimental urethral infection in the male. J. Exp. Med. 179, 911-920 (1994).

17. Schmidt, K. A., Deal, C. D., Kwan, M., Thattassery, E., & Schneider, H. Neisseria gonorrhoeae MS11 mkC opacity protein expression in vitro and during human volunteer infectivity studies. Sex. Transm. Dis. 27, 278-283 (2000).

18. Lammel, C. J. et al. Antibody-antigen specificity in the immune response to infection with Neisseria gonorrhoeae. J. Infect. Dis. 152, 990-1001 (1985).

19. Gray-Owen, S. D., Lorenzen, D. R., Haude, A., Meyer, T. F., & Dehio, C. Differential Opa specificities for CD66 receptors influence tissue interactions and cellular response to Mol. Microbiol. 26, 971-980 (1997).

20. Virji, M., Watt, S., Barker, K., Makepeace, K., & Doyonnas, R. The N-domain of the human CD66a adhesion molecule is a target for Opa proteins of Neisseria meningitidis and Neisseria gonorrhoeae. Mol. Microbiol. 22, 929-939 (1996).

21. Thompson, J. A., Grunert, F., & Zimmermann, W. Carcinoembryonic antigen gene family: molecular biology and clinical perspectives. J. Clin. Lab. Anal. 5, 344-366 (1991).

22. Beauchemin, N. et al. Redefined nomenclature for members of the carcinoembryonic antigen family. Exp. Cell Res. 252, 243-249 (1999).

23. Taylor, L. S., Paul, S. P., & McVicar, D. W. Paired inhibitory and activating receptor signals. Rev. Immunogenet. 2, 204-219 (2000).

24. Christensen, M. D. & Geisler, C. Recruitment of SHP-1 protein tyrosine phosphatase and signalling by a chimeric T-cell receptor-killer inhibitory receptor. Scand. J. Immunol. 51, 557-564 (2000).

25. Pluskota, E., Chen, Y., & D'Souza, S. E. Src homology domain 2-containing tyrosine phosphatase 2 associates with intercellular adhesion molecule 1 to regulate cell survival. J. Biol. Chem. 275, 30029-30036 (2000).

26. Muraille, E., Bruhns, P., Pesesse, X., Daeron, M., & Erneux, C. The SH2 domain containing inositol 5-phosphatase SHIP2 associates to the immunoreceptor tyrosine-based inhibition motif of Fc gammaRIIB in B cells under negative signaling. Immunol. Left. 72, 7-15 (2000).

27. Beauchemin, N. et al. Association of biliary glycoprotein with protein tyrosine phosphatase SHP-1 in malignant colon epithelial cells. Oncogene 14, 783-790 (1997).

28. Huber, M. et al. The carboxyl-terminal region of biliary glycoprotein controls its tyrosine phosphorylation and association with protein-tyrosine phosphatases SHP-1 and SHP-2 in epithelial cells. J. Biol. Chem. 274, 335-344 (1999).

29. Brummer, J., Neumaier, M., Gopfert, C., & Wagener, C. Association of pp60c-src with biliary glycoprotein (CD66a), an adhesion molecule of the carcinoembryonic antigen family downregulated in colorectal carcinomas. Oncogene 11,1649-1655 (1995).

30. Izzi, L., Turbide, C., Houde, C., Kunath, T., & Beauchemin, N. cis-Determinants in the cytoplasmic domain of CEACAM1 responsible for its tumor inhibitory function. Oncogene 18, 5563-5572 (1999).

31. Kammerer, R., Hahn, S., Singer, B. B., Luo, J. S., & von Kleist, S. Biliary glycoprotein (CD66a), a cell adhesion molecule of the immunoglobulin superfamily, on human lymphocytes: structure, expression and involvement in T cell activation. Eur. J. Immunol. 28, 3664-3674 (1998).

32. Donda, A. et al. Locally inducible CD66a (CEACAM1) as an amplifier of the human intestinal T cell response. Eur. J. Immunol. 30, 2593-2603 (2000).

33. Morales, V. M. et al. Regulation of human intestinal intraepithelial lymphocyte cytolytic function by biliary glycoprotein (CD66a). J. Immunol. 163, 1363-1370 (1999).

34. Mardiney, M., Brown, M. R., & Fleisher, T. A. Measurement of T-cell CD69 expression: a rapid and efficient means to assess mitogen- or antigen-induced proliferative capacity in normals. Cytometry 26, 305-310 (1996).

35. Vilanova, M. et al. Role of monocytes in the up-regulation of the early activation marker CD69 on B and T murine lymphocytes induced by microbial mitogens. Scand. J. Immunol. 43,155-163 (1996).

36. Chen, W. et al. Co-ligation of CD31 and Fc gamma RII induces cytokine production in human monocytes. J. Immunol. 152, 3991-3997 (1994).

37. Janssen, O. et al. Differential regulation of activation-induced cell death in individual human T cell clones. Int. Arch. Allergy Immunol. 121, 183-193 (2000).

38. Celia, M. et al. A novel inhibitory receptor (ILT3) expressed on monocytes, macrophages, and dendritic cells involved in antigen processing. J. Exp. Med. 185, 1743-1751 (1997).

39. Carretero, M. et al. Specific engagement of the CD94/NKG2-A killer inhibitory receptor by the HLA-E class Ib molecule induces SHP-1 phosphatase recruitment to tyrosine-phosphorylated NKG2-A: evidence for receptor function in heterologous transfectants. Eur. J. Immunol. 28, 1280-1291 (1998).

40. Meyaard, L. et al. LAIR-1, a novel inhibitory receptor expressed on human mononuclear leukocytes. Immunity 7, 283-290 (1997).

41. Chen, T. et al. Biliary glycoprotein (BGPa, CD66a, CEACAM1) mediates inhibitory signals. J. Leukoc. Biol. 70, 335-340 (2001).

42. Hauck, C. R., Meyer, T. F., Lang, F., & Gulbins, E. CD66-mediated phagocytosis of Opa52 Neisseria gonorrhoeae requires a Src-like tyrosine kinase- and Rac1-dependent signalling pathway. EMBO J. 17, 443-454 (1998).

43. Dennehy, K. M. et al. Thymocyte activation induces the association of phosphatidylinositol 3-kinase and pp120 with CD5. Eur. J. Immunol. 27, 679-686 (1997).

44. Janssen, O., Sanzenbacher, R., & Kabelitz, D. Regulation of activation-induced cell death of mature T-lymphocyte populations. Cell. Tissue Res. 301, 85-99 (2000).

45. Kamal, M., Katira, A., & Gordon, J. Stimulation of B lymphocytes via CD72 (human Lyb-2). Eur. J. Immunol. 21, 1419-1424 (1991).

46. Lozano, F. et al. CD5 signal transduction: positive or negative modulation of antigen receptor signaling. Crit. Rev. Immunol. 20, 347-358 (2000).

47. Perez-Villar, J. J. et al. CD5 negatively regulates the T cell antigen receptor signal transduction pathway: Involvement of SH2-containing phosphotyrosine phosphatase SHP-1. Mol. Cell. Biol. 19, 2903-2912 (1999).

48. Piali, L. et al. Murine platelet endothelial cell adhesion molecule (PECAM-1)/CD31 modulates beta 2 integrins on lymphokine-activated killer cells. Eur. J. Immunol. 23, 2464-2471 (1993).

49. Newton-Nash, D. K. & Newman, P. J. A new role for platelet-endothelial cell adhesion molecule-1 (CD31): Inhibition of TCR-mediated signal transduction. J. Immunol. 163, 682-688 (1999).

50. Adachi, T., Wakabayashi, C., Nakayama, T., Yakura, H., & Tsubata, T. CD72 negatively regulates signaling through the antigen receptor of B cells. J. Immunol. 164, 1223-1229 (2000).

51. Levine, W. C. et al. Increase in endocervical CD4 lymphocytes among women with nonulcerative sexually transmitted diseases. J. Infect. Dis. 177, 167-174 (1998).

52. McGee, Z. A., Johnson, A. P., & Taylor-Robinson, D. Pathogenic mechanisms of Neisseria gonorrhoeae: observations on damage to human fallopian tubes in organ culture by gonococci of colony type 1 or type 4. J. Infect. Dis. 143, 413-422 (1981).

53. Jerse, A. E. Experimental gonococcal genital tract infection and opacity protein expression in estradiol-treated mice. Infect. Immun. 67, 5699-5708 (1999).

54. Cohen, M. S. & Cannon, J. G. Human experimentation with Neisseria gonorrhoeae: progress and goals. J. Infect. Dis. 179(Suppl 2), S375-S379 (1999).

55. Clynes, R. et al. Modulation of immune complex-induced inflammation in vivo by the coordinate expression of activation and inhibitory receptors. J. Exp. Med. 189, 179-185.

56. Virji, M., Makepeace, K., Ferguson, D. J. P., & Watt, S. Carcinoembryonic antigens (CD66) on epithelial cells and neutrophils are receptors for Opa proteins of pathogenic neisseriae. Mol. Microbiol. 22, 941-950 (1996).

57. Hill, D. J. et al. The variable P5 proteins of typeable and non-typeable Haemophilus influenzae target human CEACAM1. Mol. Microbiol. 39, 850-862 (2001).

58. Kupsch, E.-M., Knepper, B., Kuroki, T., Heuer, I., & Meyer, T. F. Variable opacity (Opa) outer membrane proteins account for the cell tropisms displayed by Neisseria gonorrhoeae for human leukocytes and epithelial cells. EMBO J. 12, 641-650 (1993).

59. Achtman, M. et al. Purification and characterization of eight class 5 outer membrane protein variants from a clone of Neisseria meningitidis serogroup A. J. Exp. Med. 168, 507-525 (1988).

60. Dillard, J. P. & Seifert H S. A peptidoglycan hydrolase similar to bacteriophage endolysins acts as an autolysin in Neisseria gonorrhoeae. Mol. Microbiol. 25, 893-901 (1997).

61. Kunath et al., Oncogene 11:2375-2382 (1995).

62. Hsieh et al., Cancer Research 55:190-197 (1995).

63. Kleinerman et al., Cancer Research 55:1215-1220 (1995).

64. Luo et al., Oncogene 14:1697-1704 (1997).

65. Kleinerman et al., Cancer Research 56:3431-3435 (1996).

66. Bhat, K. S., C. P. Gibbs, O. Barrera, S. G. Morrison, F. Jahnig, A. Stern, E. M. Kupsch, T. F. Meyer, and J. Swanson. 1991. The opacity proteins of Neisseria gonorrhoeae strain MS11 are encoded by a family of 11 complete genes [published erratum appears in Mol Microbiol 1992 Apr;6(8):1073-6]. Mol Microbiol 5:1889-1901.

67. Bos, M. P., D. Kao, D. M. Hogan, C. C. Grant, and R. J. Belland 2002 Carcinoembryonic antigen family receptor recognition by gonococcal Opa proteins requires distinct combinations of hypervariable opa domains. Infect Immun 70:1715-1723.

68. Boulton, I. C. and S. D. Gray-Owen. 2002. Neisserial binding to CEACAM1 arrests the activation and proliferation of CD4⁺ T lymphocytes. Nat Immunol JID-100941354 3:229-236.

69. Grant, C. C., M. P. Bos, and R. J. Belland. 1999. Proteoglycan receptor binding by Neisseria gonorrhoeae MS11 is determined by the HV-1 region of OpaA. Mol Microbiol 32:233-242.

70. Gray-Owen, S. D., C. Dehio, A. Haude, F. Grunert, and T. F. Meyer. 1997. CD66 carcinoembryonic antigens mediate interactions between Opa-expressing Neisseria gonorrhoeae and human polymorphonuclear phagocytes. EMBO Journal 16:3435-3445.

71. Jacobsson, K., and L. Frykberg. 1995. Cloning of ligand-binding domains of bacterial receptors by phage display. Biotechniques 18:878-885.

72. Jacobsson, K., and L. Frykberg. 1996. Phage display shot-gun cloning of ligand-binding domains of prokaryotic receptors approaches 100% correct clones. Biotechniques 20:1070-1076.

73. Jacobsson, K., and L. Frykberg. 1998. Gene VIII-based, phage-display vectors for selection against complex mixtures of ligands. Biotechniques 24:294-301.

74. Malorny, B., G. Morelli, B. Kusecek, J. Kolberg, and M. Achtman. 1998. Sequence diversity, predicted two-dimensional protein structure, and epitope mapping of neisserial Opa proteins. J Bacteriol 180:1323-1330.

75. Sambrook, J., Fritsch, E. F., and Maniatis, T. 1989. Molecular Cloning: A Laboratory Manual. Cold Spring Harbor Laboratory Press, Cold Spring Harbor, N.Y.

76. Toleman, M., E. Aho, and M. Virji. 2001. Expression of pathogen-like Opa adhesins in commensal Neisseria: genetic and functional analysis. Mol Microbiol 3:33-44.

77. Vaz, D. 2000. Analysis of factors contributing to colonization of epidemic Canadian multi-drug resistant Staphylococcus aureus strain CMRSA. Master of Science Thesis, Department of Laboratory Medicine & Pathobiology, University of Toronto.

78. Toleman, M., E. Aho, and M. Virji 2001. Expression of pathogen-like Opa adhesins in commensal Neisseria: genetic and functional analysis. Cellular Microbiology 3:3344.

79. Webb, D. C. and A. W. Cripps 1998. Secondary structure and molecular analysis of inter-strain variability in P5 outer-membrane protein of non-typable Haemophilus influenzae isolated from diverse anatomical sites. J. Med. Microbiol. 47:1059-1067.

80. Webb, D. C. and A. W. Cripps 1999 A method for the purification and refolding of a recombinant form of the nontypeable Haemophilus influenzae P5 outer membrane protein fused to polyhistidine. Protein Expr. Purif. 15:1-7.

81. Hill, D. J., M. A. Toleman, D. J. Evans, S. Villullas, L. van Alphen, and M. Virji. 2001. Molecular Microbiology 39:850-862.

82. Virji, M., D. Evans, J. Griffith, D. Hill, L. Serino, A. Hadfield, and S. Watt 2000. Molec. Microbiol. 36:784-795.

83. Simon et al (1972) Proc. Natl. Acad, Sci USA 89:9367.

84. Remington's Pharmaceutical Sciences, Mack Publishing Company, Easton, Pa., USA 1985.

85. Handbook of Pharmaceutical Additives (compiled by Michael and Irene Ash, Gower Publishing Limited, Aldershot, England (1995).

We claim:

1. A method of modulating an immune response comprising administering an effective amount of a bacterial protein to an animal or cell in need thereof.

2. A method according to claim 1 wherein the bacterial protein can cross-link a CEACAM1 receptor.

3. A method according to claim 2 wherein the bacterial protein is from Neisseria sp. or Haemophilus sp.

4. A method according to claim 3 wherein the bacterial protein is an Opa protein.

5. A method according to claim 4 for suppressing an immune response.

6. A method according to claim 5 wherein the Opa protein is Opa₅₂ or Opa₅₇ or a fragment, analog, mimetic or derivative thereof that can suppress an immune response.

7. A method according to claim 4 wherein the Opa protein comprises a hypervariable region of an Opa protein.

8. A method according to claim 4 wherein the Opa protein comprises hypervariable region 1 (HV1) and 2 (HV2) of an Opa protein.

9. A method according to claim 5 wherein the immune response of a T lymphocyte, B lymphocyte, dendritic cell or NK cell is suppressed.

10. A method according to claim 9 wherein the T lymphocyte is a CD4+ helper T lymphocyte.

11. A method according to claim 4 for the treatment of a disease or condition selected from the group consisting of an autoimmune disease, graft rejection, graft versus host disease, an allergy, fetal loss and an inflammatory condition.

12. A method of preventing or inhibiting the growth of a tumor cell comprising administering an effective amount of a bacterial protein to an animal or cell in need thereof.

13. A method according to claim 12 wherein the bacterial protein is an Opa protein.

14. A method according to claim 13 wherein the Opa protein can cross-link a CEACAM1 receptor.

15. A method according to claim 13 wherein the Opa protein is Opa₅₂ or Opa₅₇ or a fragment, analog, mimetic or derivative thereof that can suppress an immune response.

16. A method according to claim 13 wherein the Opa protein comprises a hypervariable region of an Opa protein.

17. A method according to claim 16 wherein the Opa protein comprises hypervariable region 1 (HV1) and/or 2 (HV2) of an Opa protein.

18. A method according to claim 12 wherein the tumor cell is a T lymphocyte or B lymphocyte.

19. A method according to claim 12 further comprising administering an immune stimulatory molecule or signal.

20. A method according to claim 19 wherein the immune stimulatory molecule is IL-2.

21. A method according to claim 19 wherein the immune stimulatory signal involves activating CD3.

22. A method according to claim 1 further comprising administering an immune stimulatory molecule or signal.


SEQUENCE LISTING (14 sequences)

SEQ ID NO: 1 - DNA, 34 bp, Artificial Sequence
  SV Primer
tacgtaccatgggtgccaggcggatttagcctac

SEQ ID NO: 2 - DNA, 39 bp, Artificial Sequence
  SV Primer
tacgtaccatgggcagatgtttctgaaataatcgcttac

SEQ ID NO: 3 - DNA, 34 bp, Artificial Sequence
  HV1 Primer
tacgtaccatgggtgcgcagattatgcccgttac

SEQ ID NO: 4 - DNA, 36 bp, Artificial Sequence
  HV1 Primer
tacgtaccatgggcaggcgtggaacgtaccgttttc

SEQ ID NO: 5 - DNA, 34 bp, Artificial Sequence
  HV2 Primer
tacgtaccatgggtgccgcgtcgcctacggacac

SEQ ID NO: 6 - DNA, 33 bp, Artificial Sequence
  HV2 Primer
tacgtaccatgggcacaggttgggcgtgatgtc

SEQ ID NO: 7 - DNA, 34 bp, Artificial Sequence
  C-L Primer
tacgtaccatgggtgcgacgccgggtaccgctac

SEQ ID NO: 8 - DNA, 33 bp, Artificial Sequence
  C-L Primer
tacgtaccatgggcagaagcggtagcgcacgaa

SEQ ID NO: 9 - DNA, 34 bp, Artificial Sequence
  I-L Primer
tacgtaccatggctgccacgccgtttcttctctc

SEQ ID NO: 10 - DNA, 33 bp, Artificial Sequence
  I-L Primer
tacgtaccatgggcagccgatatagggtttgaa

SEQ ID NO: 11 - DNA, 147 bp, Artificial Sequence
  PG8ASET Phagemid Vector
ttgaaaaggaaaaacatttattcaattcgtaaactaggtgtaggtattgcatctgtaactttaggtacattacttatatctggtggcgtaacacctgctgcaaatgctgcgcaacacgatgaccatggcagtacgtacccggtgcgc

SEQ ID NO: 12 - DNA, 216 bp, Artificial Sequence
  PG8ASET Phagemid Vector
ccatggcagtacgtacccggtgcgccggtgccgtatccggacccactggaaccgcgtgcctagggatccgagggtgacgatcccgcaaaagcggcctttgactccctgcaagcctcagcgaccgaatatatcggttatgcgtgggcgatggttgttgtcattgtcggcgcaactatcggtatcaagctgtttaagaaattcacctcgaaagcaagc

SEQ ID NO: 13 - DNA, 31 bp, Artificial Sequence
  PG8-F Primer
cccggatccaatgctgcgcaacacgatgacc

SEQ ID NO: 14 - DNA, 32 bp, Artificial Sequence
  PG8-R Primer
ggggaattctgaggcttgcagggagtcaaagg